(19) United States
(12) Patent Application Publication
(10) Pub. No.: US 2025/0154482 A1
(43) Pub. Date: May 15, 2025

(54) SYSTEMS AND METHODS FOR NUCLEIC ACID DETECTION

(71) Applicant: University of Connecticut, Farmington, CT (US)

(72) Inventors: Changchun Liu, Farmington, CT (US); Jeong Moon, Farmington, CT (US)

(21) Appl. No.: 18/940,920

(22) Filed: Nov. 8, 2024

Related U.S. Application Data
(60) Provisional application No. 63/597,869, filed on Nov. 10, 2023.

(51) Int. Cl. C12N 9/22; C12N 15/11; C12Q 1/6844

(52) U.S. Cl. CPC C12N 9/22; C12N 15/111; C12Q 1/6844; C12N 2310/20

(57) ABSTRACT

The present invention relates to systems and methods for nucleic acid detection powered by CRISPR technology and in particular, an asymmetric CRISPR assay for cascade signal amplification detection of nucleic acids by leveraging the asymmetric trans-cleavage behavior of competitive crRNA.

### RELATED APPLICATION INFORMATION

This application claims priority to U.S. Application No. 63/597,869 filed on Nov. 10, 2023, the contents of which are herein incorporated by reference.

### GOVERNMENT SUPPORT

This invention was made with government support under U01CA269147, and U01AI148306 awarded by the National Institutes of Health. The government has certain rights in the invention.

### SEQUENCE LISTING STATEMENT

The contents of the electronic sequence listing titled UCONN_43790_202_SequenceListing.xml (Size: 32,202 bytes; and Date of Creation: Nov. 7, 2024) is herein incorporated by reference in its entirety.

### FIELD OF THE DISCLOSURE

Described herein are systems and methods for nucleic acid detection powered by CRISPR technology and in particular, an asymmetric CRISPR assay for cascade signal amplification detection of nucleic acids by leveraging the asymmetric trans-cleavage behavior of competitive crRNA.

### BACKGROUND

Simple, sensitive, and accurate nucleic acid detection plays a critical role in early cancer diagnostics and infectious disease detection. While PCR-based nucleic acid detection methods have been regarded as the gold-standard approach, due to their high sensitivity and specificity, there is a growing interest in developing alternative detection methods to address challenges such as the need for rapid, cost-effective, and easy-to-use diagnostic tools. One such alternative is the CRISPR (clustered regularly interspaced short palindromic repeats)-Cas (CRISPR-associated proteins) system. Originally evolved as a bacterial immune system, CRISPR-Cas has been repurposed as a powerful tool for nucleic acid detection. Various CRISPR-based methods have been developed to date, typically involving a pre-amplification step of target nucleic acids, followed by detection using CRISPR-Cas enzymes such as Cas12a or Cas13a. Although the CRISPR-Cas system offers advantages in terms of simplicity and specificity, most CRISPR-based detection tools rely on separate pre-amplification and lack quantitative detection ability, limiting their practical applications. Therefore, there is an immediate need for novel systems and methods for simplified CRISPR-based nucleic acid detection with improved sensitivity and specificity.

### SUMMARY

Described herein are systems and methods for nucleic acid detection powered by CRISPR technology.

In an aspect, disclosed is a system such as an asymmetric CRISPR assay for cascade signal amplification detection of nucleic acids including an asymmetric trans-cleavage behavior of competitive crRNA, wherein a competitive reaction between a full-sized crRNA and a split crRNA for CRISPR-Cas12a induces a cascade signal amplification, improving the target detection signal.

In an aspect, disclosed is a cascade signal amplification method for nucleic acid detection wherein an asymmetric trans-cleavage behavior of Cas12a is induced by the competitive reaction between full-sized crRNA and split crRNA (termed asymmetric CRISPR assay).

In an aspect, discloses is an asymmetric CRISPR amplification system, comprising: (a) a CRISPR-Cas protein (e.g., Cas12a); (b) at least one crisprRNA (crRNA) configured to specifically bind to a first target nucleic acid; and (c) at least one split crRNA (scrRNA) configured to specifically bind to a second target nucleic acid other than the first target nucleic acid. In another aspect, disclosed is a method for asymmetric CRISPR amplification to detect a target nucleic acid in a sample, the method comprising: (a) reacting an effective amount of a CRISPR-Cas protein, at least one crRNA, and at least one split crRNA in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex; and (b) measuring the first and second signal produced in the sample in step (a); and (c) detecting the target nucleic acid in the sample based on the first and second signal measured in step (b).

In some aspects, the at least one crRNA comprises (i) a first detectable label; and (ii) a first binding affinity to the CRISPR-cas protein. In some embodiments, the at least one scrRNA comprises: (i) a crRNA that is split into a 5′ handle region and a 3′ spacer region; (ii) a second detectable label; and (iii) a second binding affinity to the CRISPR-Cas protein that is weaker than the first binding affinity of the crRNA to the CRISPR-Cas protein. In some aspects, when the at least one crRNA, the at least one scrRNA are present in a reaction mixture with the CRISPR-Cas protein and the first and second target nucleic acids, the at least one crRNA outcompetes the at least one scrRNA to form a Cas protein/crRNA complex that is activated in the presence of the first target nucleic acid to initiate a first trans-cleavage reaction resulting in replacement of the at least one crRNA and release of the first detectable label to produce a first signal. In some aspects, upon completion of the first trans-cleavage reaction and consumption of the at least one crRNA, the at least one scrRNA forms a Cas protein/scrRNA complex that is activated in the presence of the second target nucleic acid to initiate a second trans-cleavage reaction resulting in replacement of the at least one scrRNA and release of the second detectable label to produce a second signal, thereby amplifying the first signal.

In yet another aspect, disclosed is an asymmetric CRISPR amplification system, comprising: (a) a CRISPR-Cas protein; (b) at least one single stranded DNA (ssDNA) probe comprising a 5′ region with a first detectable label and a 3′ region with a second detectable label; (c) at least one crisprRNA (crRNA) configured to simultaneously and specifically bind to the 5′ region of the ssDNA probe and a target nucleic acid; and (d) at least one split crRNA (scrRNA) configured to specifically bind to the 3′ region of the ssDNA probe. In still another aspect, disclosed is a method for asymmetric CRISPR amplification to detect a target nucleic acid in a sample, the method comprising: (a) reacting an effective amount of a CRISPR-Cas protein, at least one crRNA, at least one split crRNA, and at least one ssDNA probe in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex; (b) measuring the first and second signal produced in the sample in step (a); and (c) detecting the target nucleic acid in the sample based on the first and second signal measured in step (b).

In some aspects, (i) the at least one single stranded DNA (ssDNA) probe comprises a 5′ region with a first detectable label and a 3′ region with a second detectable label; (ii) the at least one crisprRNA (crRNA) is configured to simultaneously and specifically bind to the 5′ region of the ssDNA probe and the first target nucleic acid and comprises a first binding affinity to the CRISPR-Cas protein; and (iii) the at least one split crRNA (scrRNA) is configured to specifically bind to the 3′ region of the ssDNA probe and comprises: (1) a crRNA that is split into a 5′ handle region and a 3′ spacer region; and (2) a second binding affinity to the CRISPR-Cas protein that is weaker than the first binding affinity of the crRNA to the CRISPR-Cas protein.

In some aspects, the at least one crRNA comprises a first binding affinity to the CRISPR-Cas protein. In some aspects, the scrRNA comprises: (i) a crRNA that is split into a 5′ handle region and a 3′ spacer region; and (ii) a second binding affinity to the CRISPR-Cas protein that is weaker than the first binding affinity of the crRNA to the CRISPR-Cas protein. In some aspects, when the at least one crRNA and the at least one scrRNA are present in a reaction with the CRISPR-Cas protein, the at least one ssDNA probe, and the target nucleic acid, the at least one crRNA simultaneously binds to the 5′ region of the ssDNA probe and the target nucleic acid and outcompetes the at least one scrRNA to form a Cas protein/crRNA complex that is activated in the presence of the target nucleic acid to initiate a first trans-cleavage reaction resulting in replacement of the at least one crRNA and release of the first detectable label to produce a first signal. In some aspects, upon completion of the first trans-cleavage reaction and consumption of the crRNA, the at least one scrRNA forms a Cas protein/scrRNA complex that is activated in the presence of the at least one ssDNA probe to initiate a second trans-cleavage reaction resulting in replacement of the at least one scrRNA and release of the second detectable label to produce a second signal, thereby amplifying the first signal.

In some aspects, the at least one crRNA comprises a first binding region comprising a portion of the ′3 end of the at least one crRNA that is complementary to a portion of the 3′ end of a 5′ target strand of the target nucleic acid and a second binding region comprising a portion of the 5′ end that is complementary to a portion of the 5′ region of a 3′ target strand of the at least one ssDNA probe. In some aspects, the first binding region between the at least one crRNA and the target nucleic acid can comprise a length of from about 6 to about 15 nucleotides and the second binding region between the at least one crRNA and the at least one ssDNA probe comprises a length of from about 15 to about 6 nucleotides. In some aspects, the total length of combined first and second binding regions of the at least one crRNA can be about 21 nucleotides and as the first binding region increases in length by one nucleotide the second binding region decreases in length by one nucleotide.

In other aspects, the at least one crRNA comprises a first binding region comprising a portion of the ′3 end of the at least one crRNA that is complementary to a portion of the 5′ end of a 3′ target strand of the target nucleic acid and a second binding region comprising a portion of the 5′ end that is complementary to a portion of the 3′ region of a 5′ target strand of the at least one ssDNA probe. In other aspects, the first binding region between the at least one crRNA and the target nucleic acid can comprise a length of from about 15 to about 6 nucleotides and the second binding region between the at least one crRNA and the at least one ssDNA probe comprises a length of from about 6 to about 15 nucleotides. The total length of combined first and second binding regions of the at least one crRNA can be about 21 nucleotides and as the first binding region decreases in length by one nucleotide the second binding region increases in length by one nucleotide.

In some aspects, the at least one scrRNA comprises a binding region that is complementary to a portion of the 3′ end of a 3′ target strand of the at least one ssDNA probe. The binding region can comprise a length of from about 17 to about 24 nucleotides. The length of the binding region between the at least one scrRNA and the at least one ssDNA probe can increase by one nucleotide as the length of the second binding region between the at least one crRNA and the at least one ssDNA probe decreases in length by one nucleotide.

In other aspects, the at least one scrRNA comprises a binding region that is complementary to a portion of the 5′ end of a 5′ target strand of the at least one ssDNA probe. The binding region can comprise a length of from about 24 to 17 nucleotides. The length of the binding region between the at least one scrRNA and the at least one ssDNA probe can decrease by one nucleotide as the length of the second binding region between the at least one crRNA and the at least one ssDNA probe increases in length by one nucleotide.

In various aspects, the amount of the at least one crRNA is greater than an amount of the at least one scrRNA. The amount of the at least one crRNA can be greater than the amount of the at least one scrRNA by a factor of at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 75-fold, or at least 100-fold.

In various aspects, the first and second detectable labels comprise a fluorophore (e.g., a fluorescent dye).

In various aspects, the limit of detection of the method is at least 50 fM, at least 55 fM, at least 60 fM, at least 65 fM, at least 70 fM, at least 75 fM, at least 80 fM, at least 85 fM, at least 90 fM, at least 95 fM, or at least 100 fM. The limit of detection can be from 10 to 100 times more sensitive than a conventional CRISPR-Cas12a assay lacking at least one split scrRNA.

The systems and methods of the disclosure can be used to detect any target nucleic acid. In some aspects, the target nucleic acid comprises RNA. In some aspects, the RNA comprises miRNA. In some aspects, the miRNA comprises miR-19a, miR-21, miR-23, miR-122, miR-126, miR-146a, miR-155, miR, miR-191, or mir200a.

In various aspects, the crRNA has a length of from about 41 to about 44 nucleotides. In various aspects, the scrRNA has a length of from about 40 to about 44, wherein the 5′ handle region is from about 19 to about 20 nucleotides and the 3-spacer region is from about 21 to about 24 nucleotides.

These and other aspects and embodiments of the disclosure are described in more detail below.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIGS. 1A-B are schematic illustrations of nucleic acid detection by the conventional CRISPR assay and the asymmetric CRISPR assay. FIG. 1A is a schematic illustration of the conventional CRISPR-Cas12a assay by a single full-sized crRNA. FIG. 1B illustrates the working principle of an embodiment of an asymmetric CRISPR assay using two competitive crRNAs (a full-sized crRNA and a split crRNA). The full-sized crRNA has a higher affinity to Cas12a and specifically binds the target nucleic acid. The split crRNA is designed to bind to its own ssDNA sequence (split-T), which is different from the target nucleic acid. Due to the different binding affinities of the two crRNAs with CRISPR, CRISPR-Cas12a is first activated by the full-sized crRNA and its target nucleic acid and then is reactivated by the split RNA and its split-T, resulting in cascade signal amplification of nucleic acid detection. N.C., negative control.

FIGS. 2A-C illustrate an exemplary embodiment of a competitive CRISPR reaction between the full-sized crRNA and split crRNA. FIG. 2A illustrates the effect of the competitor crRNA (full-sized and split crRNA) on the trans-cleavage reaction by target DNA and the target-specific crRNA (full-sized and split crRNA). The competitor crRNAs were designed to bind to different target sequences than the target-specific crRNAs. Illustration was created with BioRender.com. FIG. 2B illustrates ΔFluorescence intensity (ΔFL; F_{Target DNA}−F_{Control}) of target-specific crRNA reactions in the presence of different concentrations of competitor crRNAs (10, 20, 40, and 80 nM). All the experiments were conducted in triplicate and graphs were represented by mean±standard deviation (S.D). FIG. 2C shows the results of an electrophoretic mobility shift assay. (Left) The concentration of Cy5-full-sized crRNA was fixed at 200 nM and the concentration of FAM-split-crRNA varied from 0 to 400 nM (0, 50, 100, 200, and 400 nM). The Cy5-full-sized crRNA remained bound to Cas12a even when the split crRNA concentration was increased. (Right) The concentration of FAM-split-crRNA was fixed at 200 nM and the concentration of Cy5-full-sized crRNA varied from 0 to 400 nM (0, 50, 100, 200, and 400 nM). As the concentration of Cy5-full-sized crRNA increased, FAM-split crRNA bound to Cas12a was replaced with Cy5-full-sized crRNA. Each gel image was measured by Cy5 and Alexa 488 fluorescence filters respectively, and then merged. This experiment was performed twice.

FIGS. 3A-C depict cascade signal amplification of nucleic acid detection by competitive crRNA. FIG. 3A shows an exemplary embodiment of the present disclosure in which Cas12a, full-sized crRNA, and split crRNA were incubated with different combinations of Cy5-full-T and FAM-split-T for 0, 5, 15, and 30 min. More diverse control conditions were shown in FIG. 7. This experiment was performed twice. M=marker, [Cas12a]=100 nM, [full-sized crRNA]=40 nM, [split crRNA]=10 nM, [Cy5-full-T]=100 nM, and [FAM-split-T]=50 nM. FIG. 3B shows an exemplary embodiment of the present disclosure in which Cas12a, Cy5-full-sized crRNA, and FAM-split crRNA were incubated with different combinations of full-T and split-T for 0 and 30 min. The 5′-Cy5-full-sized crRNA is trimmed at the 5′-end by LbCas12a (the first Cy5-conjugated-Uracil is cleaved). More diverse control conditions were shown in FIG. 8. This experiment was performed twice. M=marker, [Cas12a]=200 nM, [Cy5-full-sized crRNA, FAM-split crRNA handle, and split crRNA spacer]=100 nM, [full-T]=100 nM, and [split-T]=50 nM. FIG. 3C shows the results of an electrophoretic mobility shift assay. The split crRNA binding to Cas12a is inhibited by the full-sized crRNA in the absence of the full-T (left) or in the presence of non-target (middle). The split crRNA can replace the full-sized crRNA-DNA hybrid or the cleaved R-loop in Cas12a after the full-sized crRNA/Cas12a is activated by the full-T (right). This experiment was performed twice. [LbCas12a]=250 nM, [Cy5-full-sized crRNA]=200 nM, [Full-T]=100 nM, and [non-target]=100 nM. Gel was measured by the Cy5 and Alexa488 filters, respectively, and then merged.

FIGS. 4A-D illustrate a comparison and optimization of an exemplary embodiment of RNA detection of Cas12a using fragmented nucleic acid target. FIG. 4A is a schematic illustration of an exemplary embodiment of a Cas protein/crRNA complex (e.g., Cas12a/crRNA complex) binding two fragmented single-stranded nucleic acid targets, resulting in activation of a trans-cleavage reaction. The trans-cleavage reaction cannot be induced if only one of the fragmented targets is present. Illustration was created with BioRender.com. FIG. 4B, FIG. 4C, and FIG. 4D show exemplary sequences of the DNA activator (also referred to herein as a ssDNA probe) and fragmented nucleic acid target (upper FIG. 4B-FIG. 4D, underline indicates the crRNA binding region), and the corresponding fluorescence signal under different conditions (below FIG. 4B-FIG. 4D, N.C, activator, target, and the mixture of activator and target). Cas12a ribonucleoprotein complex made with DD crRNA was tested for b and d, and that made with RD crRNA was tested for c. All the experiments were conducted in triplicate and graphs were represented by mean (bold line)±standard deviation (S.D). [Cas12a]=100 nM, [DD and RD crRNA]=10 nM, [DNA activator]=100 nM, [DNA target]=100 nM, [RNA target]=100 nM.

FIGS. 5A-I illustrate the results of miRNA quantitative detection by an exemplary asymmetric CRISPR assay of the present disclosure. FIG. 5A is a schematic illustration of an exemplary miRNA detection based on the asymmetric CRISPR assay of the present disclosure. Illustration was created with BioRender.com. FIG. 5B shows the ΔFluorescence intensity (F_{Target miRNA}−F_{control}) depending on the full-sized crRNA concentration from 10 to 60 nM. [Cas12a]=100 nM, [split crRNA]=10 nM, [DNA activator]=20 nM. (n=3, Data are represented as mean±S.D of three technical replicates). FIG. 5C shows the ΔFluorescence intensity depending on the split crRNA concentration from 10 to 80 nM. [Cas12a]=100 nM, [RD crRNA]=40 nM, [DNA activator]=20 nM. (n=3, Data are represented as mean±S.D of three technical replicates). FIG. 5D shows the ΔFluorescence intensity depending on the DNA activator concentration from 10 to 80 nM. [Cas12a]=100 nM, [split crRNA]=10 nM, [RD crRNA]=40 nM. (n=3, Data are represented as mean±S.D of three technical replicates). FIG. 5E shows the ΔFluorescence intensity depending on the Cas12a concentration from 25 to 200 nM. [split crRNA]=10 nM, [RD crRNA]=40 nM, [DNA activator]=20 nM. (n=3, Data are represented as mean±S.D of three technical replicates) miR-19a sequence was used as a target, and the concentration was 10 pM. FIG. 5F illustrates the time-dependent fluorescence signal changes of the exemplary asymmetric CRISPR assay (dashed line) and Cas12a/crRNA reaction (solid line). Inset is the Δfluorescence intensity (F_{Target miRNA}−F_{control}) comparison of the asymmetric CRISPR assay (dashed line) and Cas12a/crRNA reaction (solid line). The target miR-19a concentration is 1 pM. Each curve was subtracted by the control signal. (n=7 and data represent mean±S.D of seven technical replicates). FIG. 5G illustrates the ΔFluorescence intensity of the asymmetric CRISPR assay (solid) and Cas12a/crRNA (shadowed) as a function of the target miRNA concentrations. Inset is the linear relationship between the fluorescence signal and logarithm concentration of miR-19a ranging from 1 fM to 10 pM. (n=7 and data represent mean±S.D of seven technical replicates). FIGS. 5H, and 5I show time-dependent fluorescence signal and Δfluorescence intensity of different types of miRNAs (miR-19a, let-7a, miR-21, miR-155, and miR-122). [miR-19a]=100 pM, [let-7a, miR-21, miR-155, and miR-122]=1 nM. (n=3, Data are represented as mean±S.D of three technical replicates).

FIGS. 6A-D depict the clinical application of an exemplary asymmetric CRISPR assay of the present disclosure for amplification-free miRNA detection in clinical samples. FIG. 6A is a schematic illustration of miRNA detection in human plasma samples using an exemplary asymmetric CRISPR assay. Illustration was created with BioRender.com. FIG. 6B shows the estimated target miR-19a concentrations in the bladder patient plasma samples (samples 1-10) and the healthy donor plasma samples (samples 11-15). The dashed line represents the highest target miRNA concentration of healthy donors plus standard deviation. (n=3, Data are represented as mean±S.D of three technical replicates.) FIG. 6C is a heatmap of the estimated target miR-19a concentrations of an exemplary asymmetric CRISPR assay of the present disclosure and conventional RT-qPCR assay. FIG. 6D shows miR-19a expression level analyzed by the exemplary asymmetric CRISPR assay of the present disclosure (red) and RT-qPCR (black) in bladder patients and healthy donors. The center line represents the median expression level, the bounds of the box indicate the interquartile range, and the whiskers indicate the maximum and minimum values.

FIGS. 7A-C show gel images measured after SYBR gold staining. FIG. 7A shows an example of Cas12a assembled with full-sized crRNA and split crRNA, respectively, and they were mixed with various combinations of Cy5-full-T and FAM-split-T. Gel was measured by Cy5- and Alexa488-filter and the gel images were merged. FIG. 7B shows an example of Cas12a, full-sized crRNA, and split crRNA were mixed with various combinations of Cy3-non-target and FAM-split-T. Gel was measured by Cy3- and Alexa488-filter and the gel images were merged. [LbCas12a]=100 nM, [Full-sized crRNA]=40 nM, [Split crRNA handle and spacer]=10 nM each, [Cy5-full-T]=100 nM, [Cy3-non-target]=100 nM, and [FAM-Split-T]=50 nM. FIG. 7C Gel was measured by Cy5-, Cy3-, and Alexa488-filter, and the gel images were merged. [All nucleotides]=100 nM. M=marker. These experiments were performed twice.

FIG. 8A and FIG. 8B show a gel image measured after SYBR gold staining. Gel was measured by Cy5- and Alexa488-filter and the gel images were merged. FIG. 8C shows a gel image measured after SYBR gold staining. FIG. 8D shows a gel image measured before SYBR gold staining. The reduced FAM fluorescence signal after the CRISPR-Cas12a reaction in the absence of split-T indicates pre-crRNA processing. [Cas12a]=200 nM, [Cy5-full-sized crRNA, FAM-split crRNA handle and split crRNA spacer]=100 nM, [Full-T]=100 nM, and [Split-T]=50 nM. M=marker. These experiments were performed twice.

FIG. 9A is a schematic illustration of long ssDNA detection using the asymmetric CRISPR assay. Illustration was created with BioRender.com. FIG. 9B and FIG. 9C illustrate fluorescence intensity measured after 1 h reaction depending on the concentration of long ssDNA (10 fM˜1 nM) in the presence and absence of split crRNA. (n=3, two-tailed Student's t-test; NS, not significant (P>0.1); **P<0.01, and ***P<0.001, and data represent mean±S.D of three technical replicates). The inset lines represent the limit of detection (LOD) line. LOD was calculated by the equation, mean blank+3.3×S.D of blank.

FIG. 10 illustrates ΔFluorescence intensity (F_{target DNA 10 pM}−F_{control}) under different combination of split crRNA handle and spacer. Fluorescence intensity measured after 1 h reaction. (n=3, Data represent means±S.D of three technical replicates).

FIG. 11 shows exemplary sequences of the ssRNA 5′-targets and ssDNA 3′-activators, locations of the crRNA binding site (upper a-f, underlined), and corresponding fluorescence signal under various conditions (below a-f, N.C, RNA target, DNA activator, and a mixture of RNA target and DNA activator) [Cas12a]=100 nM, [RD crRNA]=10 nM, [DNA activator]=100 nM, [RNA target]=10 nM. (n=3, Data represent means±S.D of three technical replicates).

FIG. 12 illustrates sequences of the ssDNA 5′-activators and ssRNA 3′-targets, locations of the crRNA binding site, and corresponding fluorescence signal under various conditions (below a-f, N.C, RNA target, DNA activator, and a mixture of RNA target and DNA activator). [Cas12a]=100 nM, [DD crRNA]=10 nM, [DNA activator]=100 nM, [RNA target]=100 nM. (n=3, Data represent means±S.D of three technical replicates).

FIG. 13 illustrates sequences of the ssDNA 5′-activators and ssDNA 3′-targets, locations of the crRNA binding site, and corresponding fluorescence signal under various conditions (below a-f, N.C, DNA target, DNA activator, and a mixture of DNA target and DNA activator). [Cas12a]=100 nM, [DD crRNA]=10 nM, [DNA activator]=100 nM, [DNA target]=100 nM. (n=3, Data represent means±S.D of three technical replicates).

FIG. 14B shows the real-time fluorescence signal of Cas12a/crRNA reaction using target miR-19a in the concentration range of 100 fM to 1 nM. FIG. 14B shows the fluorescence signal measured after 1 h reaction. The limit of detection (LOD) was calculated by the equation, mean blank+3.3×S.D of a blank and was estimated to be 1 pM. (n=3, two-tailed Student's t-test; NS, not significant (P>0.1); *P<0.05, data represent mean±S.D of three technical replicates).

FIGS. 15A-C show a comparison of an exemplary Cas12a/crRNA trans-cleavage activity between a split target (ssRNA and ssDNA) and long ssDNA target containing identical sequences of split ssRNA and ssDNA. FIG. 15A is a schematic illustration of an exemplary CRISPR detection for split target (ssRNA and ssDNA) and long ssDNA. FIG. 15B shows a real-time fluorescence signal and endpoint fluorescence intensity depending on the concentration (100 fM, 1 pM, and 10 pM) of split ssRNA. FIG. 15C shows a real-time fluorescence signal and endpoint fluorescence intensity depending on the concentration (100 fM, 1 pM, and 10 pM) of long ssDNA target. Experiments were conducted three times and data represent mean±S.D of three technical replicates (two-tailed Student's t-test; NS, not significant (P>0.1); *P<0.05, **P<0.01, and ***P<0.001). The inset lines represent the limit of detection (LOD) line. LOD was calculated by the equation, mean blank+3.3×S.D of blank. The LOD of both targets was calculated as 1 pM, respectively. [Cas12a]=100 nM, [crRNA]=10 nM, and [split ssDNA]=10 nM.

FIG. 16 shows a real-time fluorescence signal and the fluorescence intensity measured after 1 h reaction of (a) an exemplary CRISPR-Cas12a assay of the present disclosure without split crRNA and (b) the asymmetric CRISPR assay. (n=7, two-tailed Student's t-test; NS, not significant (P>0.1); *P<0.05, **P<0.01, and ***P<0.001; data represent mean±S.D of seven technical replicates). The inset lines represent the limit of detection (LOD) line. LOD was calculated by the equation, mean blank+3.3×S.D of blank.

FIG. 17 shows the fluorescence signal of let-7a detection using (a) an exemplary Cas12a/crRNA assay of the present disclosure without split crRNA and (b) an asymmetric CRISPR assay. The fluorescence signal was measured after 1 h incubation. The inset dashed lines represent the limit of detection (LOD) line. LOD was calculated by the equation of mean blank+3.3×S.D of blank. (n=3, two-tailed Student's t-test; NS, not significant (P>0.1); *P<0.05, **P<0.01, and ***P<0.001; data represent mean±S.D of three technical replicates).

FIG. 18A shows real-time fluorescence responses of standard target miR-19a by RT-qPCR. FIG. 18B shows the linear relationship between the Cq and logarithm of miR-19a concentration. The linear regression equation is y=−3.572x−17.103 (R²=0.999). n=3, data represent mean±S.D of three technical replicates.

### DETAILED DESCRIPTION

Disclosed herein are the systems and methods such as an asymmetric CRISPR assay for cascade signal amplification detection of nucleic acids in a subject such as mammals, in particular humans.

Recently, many efforts have aimed to simplify CRISPR-based nucleic acid detection and improve its sensitivity and specificity. New strategies employ multiple crRNAs or catalytic nucleic acid circuits to avoid additional manipulation, yet these approaches require long targets capable of binding multiple crRNAs and have a long detection time. Several studies have shown that chemically modified crRNA can promote improved gene correction compared to unmodified crRNA. For example, some researchers reported that RNA-DNA hybrid crRNA and extensions on the 5′ or 3′ end of crRNA can activate the catalytic efficiency of CRISPR, thus enhancing the sensitivity and specificity of CRISPR-based nucleic acid detection. Another study reported that a split crRNA containing separated scaffold and spacer RNA can catalyze highly specific cis-cleavage in Cas12a and, however, did not explore its effect on trans-cleavage.

CRISPR-Cas12a is an RNA-guided DNA endonuclease that can be programmed to cleave target DNA and has been harnessed for gene editing since its discovery. In addition, Cas12a can induce indiscriminate cleavage of non-target single-stranded DNA (ssDNA) after target-specific recognition and cleavage, which is referred to as trans-cleavage activity. Because of this property, Cas12a has been widely applied for nucleic acid-based molecular diagnostics. However, since current Cas12a-based detection methods are limited to detecting double-stranded DNA (dsDNA) or ssDNA, a reverse transcription step and/or additional DNA pre-amplification steps are required for RNA detection. In addition, CRISPR-Cas12a is typically activated by dsDNA target with a protospacer adjacent motif (PAM).

In an aspect, disclosed is a system such as an asymmetric CRISPR assay for cascade signal amplification detection of nucleic acids by leveraging the asymmetric trans-cleavage behavior of competitive crRNA. The examples of the instant application demonstrate that the competitive reaction between a full-sized crRNA and split crRNA for CRISPR-Cas12a can induce cascade signal amplification, significantly improving the target detection signal. In an embodiment, CRISPR-Cas12a can recognize fragmented RNA/DNA targets, enabling direct RNA detection by Cas12a. In an embodiment, the asymmetric CRISPR assay quantitatively detected microRNA without the need for pre-amplification, achieving a detection sensitivity of about 856 aM (attomolar). Moreover, using the system and method disclosed herein, miR-19a biomarker was analyzed and quantified in plasma samples from bladder cancer patients. This asymmetric CRISPR assay can be widely applied for simple and sensitive nucleic acid detection in various diagnostic settings.

The examples of the instant application investigated asymmetric trans-cleavage behavior of Cas12a induced by the competitive reaction between full-sized crRNA and split crRNA and developed a cascade signal amplification method for nucleic acid detection, termed asymmetric CRISPR assay. An asymmetric trans-cleavage reaction as well as a conformational resetting of the CRISPR enzyme resulting from competition between two types of crRNAs was observed.

The examples of the instant application demonstrate that this competitive CRISPR reaction could significantly enhance the detection sensitivity of CRISPR proteins such as CRISPR-Cas12a without the need for an additional DNA amplification step. Any suitable CRISPR-Cas protein disclosed herein can be used. In an embodiment, Cas12a can recognize fragmented RNA/DNA targets, enabling direct detection of RNA. In an embodiment, an asymmetric CRISPR assay is applied to quantitatively detect a microRNA (miRNA) biomarker for liquid biopsy, demonstrating an amplification-free detection approach using CRISPR-Cas12a. Notably, this asymmetric CRISPR assay uses a single CRISPR-Cas12a enzyme and can achieve highly sensitive detection of miRNA, thereby showing potential as a powerful tool for simple, rapid, and highly sensitive nucleic acid-based molecular diagnostics.

In an embodiment, the CRISPR reaction significantly enhances the detection sensitivity of CRISPR-Cas12a without the need for an additional DNA amplification step. In an embodiment, the competitive reaction between full-sized crRNA and split crRNA for CRISPR-Cas12a can induce cascade signal amplification, significantly improving the target detection signal. In an embodiment, the CRISPR-Cas12a can recognize fragmented RNA/DNA targets, enabling direct RNA detection by Cas12a. In an embodiment, the asymmetric CRISPR assay directly detects microRNA without the need for pre-amplification, achieving an at attomolar-level detection sensitivity. In an embodiment, there is a competitive CRISPR reaction between full-sized crRNA and split crRNA, that improves the detection sensitivity of CRISPR-Cas12a. In an embodiment, an asymmetric CRISPR assay for cascade signal amplification detection of nucleic acid is applied to directly detect miRNA in clinical cancer samples without the need for pre-amplification or reverse transcription. In an embodiment, the asymmetric CRISPR assay uses two sets of competitive crRNAs: i) a target-specific full-sized crRNA and ii) an independent split crRNA. In an embodiment, the crRNAs have different binding affinities to CRISPR-Cas12a.

In an aspect, disclosed is a cascade signal amplification method for nucleic acid detection wherein an asymmetric trans-cleavage behavior of Cas12a is induced by the competitive reaction between full-sized crRNA and split crRNA (termed asymmetric CRISPR assay).

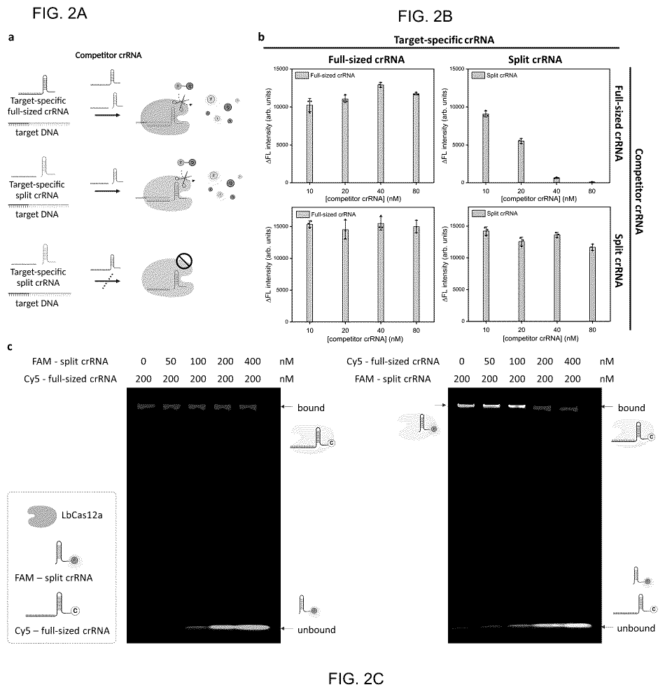

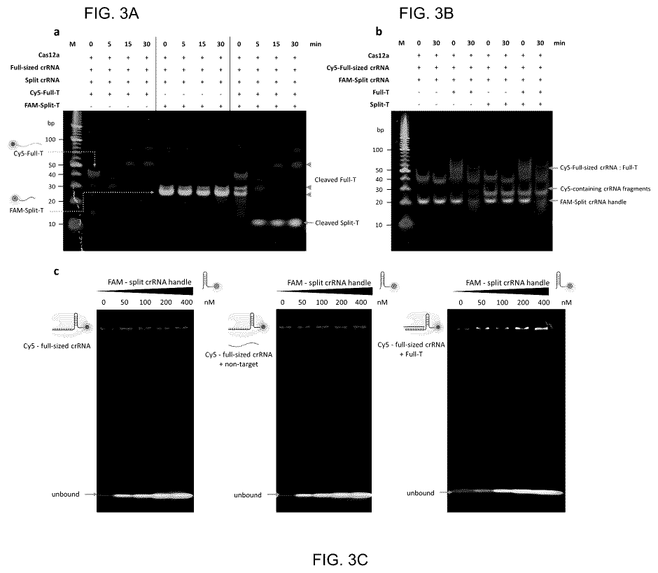

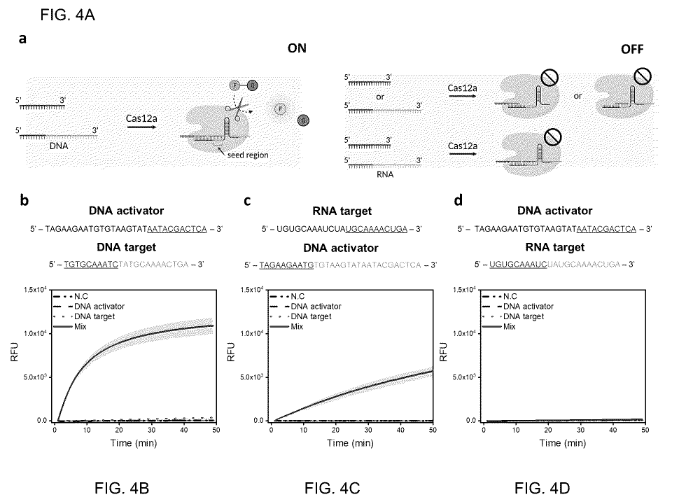

The examples of the instant application demonstrate two major CRISPR findings in this study: (i) the competitive reaction between full-sized crRNA and split crRNA can improve the detection sensitivity of CRISPR-Cas12a (FIG. 2 and FIG. 3), and (ii) Cas12a can recognize fragmented RNA/DNA targets that are complementary to the crRNA, enabling direct detection of RNA (FIG. 4). Based on these findings, an asymmetric CRISPR assay was developed for cascade signal amplification detection of nucleic acid and applied it to quantitatively detect miRNA in clinical cancer samples without the need for pre-amplification or reverse transcription.

It is reported that split crRNA can catalyze highly specific and efficient cleavage of target DNA by Cas12a nucleases in vitro and in lysates of human cells. However, little is known about the trans-cleavage activity of split crRNA. In this study, an interesting trans-cleavage behavior between split crRNA and full-sized crRNA was found. The results obtained performing the examples described herein indicate that full-sized crRNA has a stronger binding affinity to Cas12a than split crRNA, which enables full-sized crRNA to regulate the split crRNA reaction. Specifically, when two types of crRNAs with different affinities to Cas12a are mixed, the trans-cleavage reaction caused by the crRNA showing a stronger affinity for Cas12a is fast and dominant. By contrast, the reaction induced by the crRNA with a weaker affinity for Cas12a is slow but helps to enhance the sensitivity of the dominant reaction. Leveraging these unique asymmetric trans-cleavage behaviors of competitive crRNA, a signal amplification method that can improve the sensitivity of CRISPR simply by adding split crRNA and its target ssDNA was developed. It is believed that this is the first report of a signal amplification method using competitive crRNAs with different CRISPR reactivities.

The class-II type V CRISPR-Cas12a system is known to recognize dsDNA and ssDNA activators and exhibits collateral nonspecific ssDNA cleavages. In this study, it was revealed that Cas12a can directly recognize RNA targets when the RNA is placed at the 3′ end of the crRNA and supported by a DNA located at the 5′ end of the crRNA. In particular, the binding region of the RNA to the crRNA is important, as the ssDNA must be located at the 5′ end of the crRNA, a seed region that plays a critical role in target recognition and cleavage by the Cas12a protein. It has been demonstrated that Cas12a can be programmed to bind ssRNA, supporting the findings described herein. More importantly, these results indicate that Cas12a can be programmed to detect both RNA and DNA.

Furthermore, the asymmetric CRISPR assay was adapted to quantitatively detect miRNA biomarker by combining fragmented RNA/DNA targets. In the assay, the split crRNA was designed to bind a DNA activator that supports RNA detection; as a result, target miRNA was selectively detected with a LOD of 856 aM (1,000-fold more sensitive than the CRISPR detection without split crRNA), in line with previous studies combining Cas12a with pre-amplification steps. Moreover, it was demonstrated that the assay can specifically detect miRNA in human plasma samples, showing its potential application for liquid biopsy. Compared with the current CRISPR-Cas system-based miRNA detection methods (Table 1), the developed method is a one-pot, one-step isothermal signal amplification method using a single CRISPR enzyme, enabling simple, sensitive, and quantitative detection of nucleic acids.

TABLE 1
Comparison of asymmetric CRISPR assay with other miRNA
detection methods using CRISPR/Cas12a system

| | | One-pot | # of major | |
|---|---|---|---|---|
| Method | Mechanism | reaction | components | LOD |
| PCDetection | polyA-tailing + cDNA | No, 3 | 4 enzymes, | miR-299: 50 fM |
| | synthesis + Recombinase | steps | 3 probes | |
| | polymerase amplification | | | |
| | (RPA) + Cas12a | | | |
| CRISPR-HCR | Hybridization chain | No, 2 | 1 enzyme, | miR-21: 1 fM |
| | reaction (HCR) + Cas12a | steps | 3 probes | |
| EXTRA- | Rolling-circle | Yes | 3 enzymes, | miR-21: 1.64 |
| CRISPR | amplification (RCA) + | | 3 probes | fM; miR-196a: |
| | Cas12a | | | 1.35 fM |
| CRISPR- | Catalytic hairpin assembly | No, 2 | 1 enzyme, | miR-141: 0.14 |
| CHA | (CHA) + Cas12a | steps | 2 probes | fM; miR-155: |
| | | | | 0.15 fM |
| ccTdT- | Click chemistry ligation + | No, 4 | 2 enzymes, | miR-21: 88 fM |
| Cas12a | Magnetic separation + | steps | 2 probes | |
| | polyT-tailing + Cas12a | | | |
| CAL-LAMP | Ligation + Loop-mediated | No, 3 | 3 enzymes, | Let-7a: 0.1 fM |
| | Isothermal Amplification | steps | 6 probes | |
| | (LAMP) + Cas12a | | | |
| Cas12a- | Ligation + Rolling-circle | No, 3 | 3 enzymes, | miR-21: 34.7 fM |
| enhanced | amplification (RCA) + | steps | 3 probes | |
| RCA | Cas12a | | | |
| Asymmetric | Cascade signal | Yes | 1 enzyme, | miR19a: 856 aM |
| CRISPR | amplification based on the | | Split | |
| | competitive reaction | | crRNA, | |
| | between Full-sized crRNA | | 1 activator | |
| | and split crRNA | | | |

The asymmetric CRISPR assay can be further expanded and improved from several aspects, enabling point-of-care testing in resource-limited environments. First, to simplify the manual operation, the assay can be further integrated into a microfluidic platform for automated sample preparation and liquid manipulation. Second, to improve single-nucleotide sequence discrimination of the assay, newly engineered CRISPR enzymes and engineered crRNAs can be exploited and optimized. Third, to eliminate the need for expensive fluorescence detection equipment, a simple and affordable smartphone detector can be developed for fluorescence recording and signal processing. Last, beyond the current miRNA detection for early cancer diagnostics, the asymmetric CRISPR assay can be further developed to detect other nucleic acid biomarkers of infectious diseases, such as SARS-CoV-2, and HIV. Overall, disclosed CRISPR-Cas12a reaction system presents a powerful nucleic acid detection approach with the potential for clinical applications for early cancer diagnostics and infectious disease detection.

In an aspect, the present disclosure provides an asymmetric CRISPR amplification system, comprising: (a) a CRISPR-Cas protein; (b) at least one crisprRNA (crRNA) configured to specifically bind to a first target nucleic acid; and (c) at least one split crRNA (scrRNA) configured to specifically bind to a second target nucleic acid other than the first target nucleic acid.

In a related aspect, the present disclosure provides a method for asymmetric CRISPR amplification to detect a target nucleic acid in a sample, the method comprising: (a) reacting an effective amount of a CRISPR-Cas protein, at least one crRNA, and at least one split crRNA in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex; and (b) measuring the first and second signal produced in the sample in step (a); and (c) detecting the target nucleic acid in the sample based on the first and second signal measured in step (b).

In another aspect, the present disclosure provides an asymmetric CRISPR amplification system, comprising: (a) a CRISPR-Cas protein; (b) at least one single stranded DNA (ssDNA) probe comprising a 5′ region with a first detectable label and a 3′ region with a second detectable label; (c) at least one crisprRNA (crRNA) configured to simultaneously and specifically bind to the 5′ region of the ssDNA probe and a target nucleic acid; and (d) at least one split crRNA (scrRNA) configured to specifically bind to the 3′ region of the ssDNA probe.

In a related aspect, the present disclosure provides a method for asymmetric CRISPR amplification to detect a target nucleic acid in a sample, the method comprising: (a) reacting an effective amount of a CRISPR-Cas protein, at least one crRNA, at least one split crRNA, and at least one ssDNA probe in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex; (b) measuring the first and second signal produced in the sample in step (a); and (c) detecting the target nucleic acid in the sample based on the first and second signal measured in step (b).

### Cas Proteins

The disclosure contemplates the use of any Cas protein in the asymmetric CRISPR amplification systems and related methods. In some embodiments, the CRISRS-Cas protein is a Cas protein from a Type I system. Exemplary Type I system Cas proteins include, without limitation, Cas3, Cse1, Cas5, Cas6, Cas7 and Cas8. In other embodiments, the Cas protein is a Type II system Cas protein. Exemplary Type II Cas proteins include, without limitation, Cas9, Cas12, Cas12a, Cas12b, Cas13, Cas13a, Cas13b, Cas13c, Cas13d, and Cas14. In yet other embodiments, the CRISPR-Cas protein is a Cas protein from a Type III system. Exemplary Type II system Cas proteins include, without limitation, Cas4, Cas10, Csm and Cmr complexes, and Csx. In still other embodiments, the CRISPR-Cas protein is CasX, CasY, or CasPhi.

CRISPR-RNA (crRNA) and Split CRISPR-RNA (scrRNA)

Aspects of the present disclosure involve the use of crRNAs and scrRNAs that can be configured to specifically bind to the same target nucleic acid or to different target nucleic acids.

In some aspects, the systems and methods of the disclosure contemplate the use of at least one crRNA that is configured to specifically bind to at least one first target nucleic acid and at least one scrRNA that is configured to specifically bind to at least one second target nucleic acid other than the first target nucleic acid of the at least one crRNA.

The skilled artisan will appreciate that number of crRNAs and scrRNAs used in any application can vary, as can their length and sequence, depending on a variety of factors, including the targets of the crRNAs and scrRNAs. In some embodiments, the at least one crRNA comprises at least one, at least two, at least three, at least four, or at least five crRNAs designed to specifically bind to at least one, at least two, at least three, at least four, or at least five target nucleic acids and the at least one scrRNA comprises at least one, at least two, at least three, at least four, or at least five scrRNAs designed to specifically bind to at least one, at least two, at least three, at least four, or at least five target nucleic acids other than the targets of the at least one crRNAs. The crRNAs can be designed to target different nucleic acids or multiple crRNAs can be designed to target different portions of the same nucleic acid.

In other aspects, the systems and methods of the disclosure contemplate the use of at least one single stranded DNA (ssDNA) probe comprising a 5′ region with a first detectable label and a 3′ region with a second detectable label and the at least one crRNA is configured to simultaneously and specifically bind to the 5′ region of the ssDNA probe and a target nucleic acid and the at least one scrRNA is configured to specifically bind to the 3′ region of the ssDNA probe.

In some embodiments of aspects utilizing at least one ssDNA probe, the at least one crRNA can comprise a first binding region comprising a portion of the ′3 end of the at least one crRNA that is complementary to a portion of the 3′ end of a 5′ target strand of the target nucleic acid and a second binding region comprising a portion of the 5′ end that is complementary to a portion of the 5′ region of a 3′ target strand of the at least one ssDNA probe. In other embodiments of aspects utilizing at least one ssDNA probe, the at least one crRNA comprises a first binding region comprising a portion of the ′3 end of the at least one crRNA that is complementary to a portion of the 5′ end of a 3′ target strand of the target nucleic acid and a second binding region comprising a portion of the 5′ end that is complementary to a portion of the 3′ region of a 5′ target strand of the at least one ssDNA probe.

### Cascade Signal Amplification

The systems and methods of the disclosure leverage the competitive binding of crRNAs and split crRNAs (scrRNAs) to initiate sequential trans-cleavage reactions by the CRISPR-Cas protein resulting in a cascade amplification of the signal. Accordingly, in some embodiments, the at least one crRNA comprises a binding affinity to the CRISPR-Cas protein that is stronger than the binding affinity of the at least one scrRNA to the CRISPR-Cas protein.

In some aspects, when the at least one crRNA and the at least one scrRNA are present in a reaction mixture with the CRISPR-Cas protein and the first and second target nucleic acids, the at least one crRNA outcompetes the at least one scrRNA to form a Cas protein/crRNA complex that is activated in the presence of the first target nucleic acid to initiate a first trans-cleavage reaction resulting in replacement of the at least one crRNA and release of the first detectable label to produce a first signal. Upon completion of the first trans-cleavage reaction and consumption of the at least one crRNA, the at least one scrRNA forms a Cas protein/scrRNA complex that is activated in the presence of the second target nucleic acid to initiate a second trans-cleavage reaction resulting in replacement of the at least one scrRNA and release of the second detectable label to produce a second signal, thereby amplifying the first signal.

In other aspects, when the at least one crRNA and the at least one scrRNA are present in a reaction with the CRISPR-Cas protein, the at least one ssDNA probe, and the target nucleic acid, the at least one crRNA simultaneously binds to the 5′ region of the ssDNA probe and the target nucleic acid and outcompetes the at least one scrRNA to form a Cas protein/crRNA complex that is activated in the presence of the target nucleic acid to initiate a first trans-cleavage reaction resulting in replacement of the at least one crRNA and release of the first detectable label to produce a first signal. Upon completion of the first trans-cleavage reaction and consumption of the crRNA, the at least one scrRNA forms a Cas protein/scrRNA complex that is activated in the presence of the at least one ssDNA probe to initiate the second trans-cleavage reaction resulting in replacement of the at least one scrRNA and release of the second detectable label to produce a second signal, thereby amplifying the first signal.

In addition to leveraging relative binding affinities between the at least one crRNA and at least one scrRNA and the CRISPR-Cas protein system to initiate sequential trans-cleavage reactions and amplify detectable signals, the systems and methods of the present disclosure contemplate utilizing greater amounts of at least one crRNA and lesser amounts of at least one scrRNA in a sample to facilitate the sequential trans-cleavage reactions. Accordingly, in some aspects, an amount of the at least one crRNA is greater than an amount of the at least one scrRNA. The amount of the at least one crRNA can be greater than the amount of the at least one scrRNA by a factor of at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 75-fold, or at least 100-fold.

### Detectable Labels

The polynucleotides of the present disclosure (e.g., crRNAs, scrRNAs, ssDNA probes) can be designed to include a detectable label. The detectable label can be positioned anywhere within the polynucleotide sequence of the at least one crRNA, at least one scrRNA, or at least one ssDNA probe. In some embodiments, the at least one crRNA comprises a detectable label at the 5′ end. In other embodiments, the at least one crRNA comprises a detectable label at the 3′ end. In yet other embodiments, the at least one crRNA comprises a detectable label in the first binding region. In still other embodiments, the at least one crRNA comprises a detectable label in the second binding region.

In some embodiments, the at least one scrRNA comprises a detectable label at the 5′ end. In other embodiments, the at least one scrRNA comprises a detectable label at the 3′ end. In still other embodiments, the at least one scrRNA comprises a detectable label in the 5′ handle region. In some instances, the detectable label is positioned at the 5′ end of the 5′ handle region. In other instances, the detectable label is positioned at the 3′ end of the 5′ handle region. In still other instances, the 5′ handle region can have a detectable label positioned at both the 5′ end and the 3′ end of the handle region. In yet other embodiments, the at least one scrRNA comprises a detectable label in the 3′ spacer region. In some instances, the detectable label is positioned at the 5′ end of the 3′ spacer region. In other instances, the detectable label is positioned at the 3′ end of the 3′ spacer region. In still other instances, a detectable label is positioned at both the 3′ end and the 5′ end of the 3′ spacer region.

The Cas protein\crRNA and Cas proteins\crRNA complexes generate measurable signals by releasing detectable labels during the replacement of the crRNAs and scrRNAs in the trans-cleavage reactions, resulting in cascade amplification of the signals.

The asymmetric CRISPR amplification systems and methods of the present disclosure contemplate using any methods for measuring and detecting the first and second signals generated respectively by the detectable labels cleaved from the crRNAs and scrRNAs.

### Target Nucleic Acids

The asymmetric CRISPR amplification systems and methods of the present disclosure can be used to detect any target nucleic acid of interest. In some aspects, the target nucleic acid comprises DNA. In other aspects, the target nucleic acid comprises RNA. In still other aspects, the target nucleic comprises DNA and RNA.

In yet other aspects, the RNA comprises miRNA. The asymmetric CRISPR amplification systems and methods of the present disclosure can be designed to detect any miRNA. Exemplary miRNA targets include, without limitation, miR-19a, miR-21, miR-23, miR-122, miR-126, miR-146a, miR-155, miR, miR-191, mir200a, or any combination thereof.

### Kits

Aspects of the disclosure contemplate kits comprising an asymmetric CRISPR amplification system of the present disclosure for implementing related methods of the disclosure. In an aspect, the present disclosure provides a kit comprising an asymmetric CRISPR amplification system, the kit comprising: (a) a CRISPR-Cas protein; (b) at least one crisprRNA (crRNA) configured to specifically bind to a first target nucleic acid; and (c) at least one split crRNA (scrRNA) configured to specifically bind to a second target nucleic acid other than the first target nucleic acid.

In a related aspect, the present disclosure provides a kit for implementing a method for asymmetric CRISPR amplification to detect a target nucleic acid in a sample. The kit includes instructions for performing the method. For example, the kit includes instructions for (a) reacting an effective amount of a CRISPR-Cas protein, at least one crRNA, and at least one split crRNA in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex; and (b) measuring the first and second signal produced in the sample in step (a); and (c) detecting the target nucleic acid in the sample based on the first and second signal measured in step (b).

In another aspect, the present disclosure provides a kit comprising an asymmetric CRISPR amplification system, the kit comprising: (a) a CRISPR-Cas protein; (b) at least one single stranded DNA (ssDNA) probe comprising a 5′ region with a first detectable label and a 3′ region with a second detectable label; (c) at least one crisprRNA (crRNA) configured to simultaneously and specifically bind to the 5′ region of the ssDNA probe and a target nucleic acid; and (d) at least one split crRNA (scrRNA) configured to specifically bind to the 3′ region of the ssDNA probe.

In a related aspect, the present disclosure provides a kit for implementing a method for asymmetric CRISPR amplification to detect a target nucleic acid in a sample. The kit includes instructions for performing the method. For example, the kit includes instructions for (a) reacting an effective amount of a CRISPR-Cas protein, at least one crRNA, at least one split crRNA, and at least one ssDNA probe in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex; (b) measuring the first and second signal produced in the sample in step (a); and (c) detecting the target nucleic acid in the sample based on the first and second signal measured in step (b).

### Uses

The asymmetric CRISPR amplification systems and methods of the present disclosure can be used for a variety of purposes, including research and clinical applications that involve detecting target nucleic acids in a sample. Aspects of the present disclosure involve using the asymmetric CRISPR amplification systems and methods to detect diseases, conditions, and disorders associated with altered levels of expression of target nucleic acids (e.g., disease-associated miRNAs, e.g., cancer-associated miRNAs).

In an aspect, the disclosure provides a method for detecting miRNA in a biological sample obtained from a subject having or suspected of having cancer (e.g., bladder cancer), the method comprising: (a) obtaining a biological sample from the subject; (b) contacting the biological sample with an asymmetric CRISPR amplification system of the present disclosure for a period of time sufficient for the CRISPR amplification system to bind to the miRNA in the sample and initiate sequential trans-cleavage reactions that produce an amplified signal in the sample; and (c) detecting the miRNA in the sample based on a measurement of the amplified signal in the sample. In some embodiments, the CRISPR amplification system comprises (a) a CRISPR-Cas protein; (b) at least one single stranded DNA (ssDNA) probe comprising a 5′ region with a first detectable label and a 3′ region with a second detectable label; (c) at least one crisprRNA (crRNA) configured to simultaneously and specifically bind to the 5′ region of the ssDNA probe and the target nucleic miRNA in the sample; and (d) at least one split crRNA (scrRNA) configured to specifically bind to the 3′ region of the ssDNA probe. In other embodiments, the CRISPR amplification system comprises (a) a CRISPR-Cas12a protein; (b) at least one single stranded DNA (ssDNA) probe comprising a 5′ region with a first detectable label and a 3′ region with a second detectable label; (c) at least one crisprRNA (crRNA) configured to simultaneously and specifically bind to the 5′ region of the ssDNA probe and the target nucleic miRNA in the sample; and (d) at least one split crRNA (scrRNA) configured to specifically bind to the 3′ region of the ssDNA probe.

In some embodiments, the miRNA comprises miR-19a. In some embodiments, the miRNA comprises miR-21. In some embodiments, the miRNA comprises miR-23. In some embodiments, the miRNA comprises miR-122. In some embodiments, the miRNA comprises miR-126. In some embodiments, the miRNA comprises miR-146a. In some embodiments, the miRNA comprises miR-155. In some embodiments, the miRNA comprises miR-191. In some embodiments, the miRNA comprises mir200a. In some embodiments, the sample is a plasma sample. In some embodiments, the sample is a serum sample. In some embodiments, the sample is obtained by liquid biopsy. The detection of the miRNA in the sample obtained from the patient can be used for the diagnosis (e.g., early detection) and/or prognosis of the cancer in the subject. For example, the levels of the miRNA can be detected in samples obtained from the subject at different time points, and the development and progression of the cancer and/or efficacy of treatment of the cancer can be assessed based on an increase or decrease of the levels of miRNA detected in the subject's sample.

The methods of detecting miRNA are not limited to detecting miRNAs associated with any particular type of cancer. In some embodiments, the cancer is bladder cancer. In some embodiments, the bladder cancer is urothelial carcinoma (e.g., urothelial papillary carcinoma. In some embodiments, the bladder cancer is squamous cell carcinoma. In some embodiments, the bladder cancer is adenocarcinoma. In some embodiments, the bladder cancer is small cell carcinoma. In some embodiments, the bladder cancer is a sarcoma.

The skilled artisan will appreciate that the sequences of the at least one crRNA, at least one scrRNA, at least one ssDNA probe, and/or miRNA used in the method for detecting the miRNA in the subject's sample can be sequences listed in Table 3.

In another aspect, the system is used to detect a target sequence or a target nucleic acid. In an embodiment, the target sequence includes SARS-CoV-2, influenza virus, drug-resistant influenza viruses, human immunodeficiency virus (HIV), and/or a combination thereof. In an embodiment, the target sequence includes several types of biomarkers such as miRNA, mRNA, circulating cell-free DNA (cfDNA), RNA (cfRNA), and/or a combination thereof. In an embodiment, the target sequence is a SARS-CoV-2 sequence. In an embodiment, the target sequence is a human immunodeficiency virus (HIV).

### Assay Performance

The asymmetric CRISPR amplification systems and methods of the present disclosure exhibit improved sensitivity compared to conventional CRISPR-Cas protein nucleic acid detection assays that do not leverage competitive binding affinities between crRNAs and scrRNAs to the CRISPR-Cas protein complexes.

In various aspects, the CRISPR amplification systems and methods of the present disclosure exhibit improved limits of detection. In some embodiments, the limit of detection of is at least 50 fM, at least 55 fM, at least 60 fM, at least 65 fM, at least 70 fM, at least 75 fM, at least 80 fM, at least 85 fM, at least 90 fM, at least 95 fM, or at least 100 fM. In other embodiments, the limit of detection is about 10 to 100 times more sensitive than conventional CRISPR-Cas protein assays lacking at least one split scrRNA.

The CRISPR amplification systems and methods of the present disclosure can be performed in the absence of pre-amplification of target nucleic acids.

The present disclosure is illustrated and further described in more detail with reference to the following non-limiting examples. Section headings as used in this section and the entire disclosure herein are merely for organizational purposes and are not intended to be limiting.

### EXAMPLES

### Example 1—Asymmetric CRISPR Assay

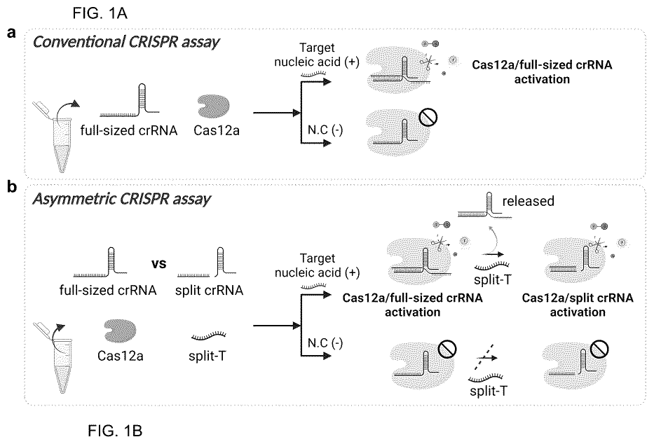

FIG. 1 illustrates the working principle of the asymmetric CRISPR assay for cascade signal amplification detection of nucleic acid targets. Unlike the conventional CRISPR-Cas12a assay, which uses a single full-sized crRNA (FIG. 1a), the asymmetric CRISPR assay uses two sets of competitive crRNAs: i) a target-specific full-sized crRNA and ii) an independent split crRNA (FIG. 1b). The full-sized crRNA is designed to specifically bind to target nucleic acids (full-T). The split crRNA consists of a separate 5′-scaffold moiety (hereafter referred to as ‘handle’) and a 3′-spacer moiety (hereafter referred to as ‘spacer’). The split crRNA is designed to specifically bind its own independent ssDNA (split-T) with a different sequence from the target nucleic acid. In the asymmetric CRISPR assay, when the two crRNAs (full-sized crRNA and split crRNA) and the split-T are mixed in one-pot, the full-sized crRNA will first form the Cas12a/full-sized crRNA complex because it has a stronger binding affinity to Cas12a compared with the split crRNA. Thus, the high-affinity full-sized crRNA inhibits the binding of the split crRNA to Cas12a and CRISPR-Cas12a cannot be activated even in the presence of the split-T (FIG. 1b). In the presence of target nucleic acids, the Cas12a/full-sized crRNA complex is specifically activated and initiates the first trans-cleavage reaction. Subsequently, the split crRNA can replace the full-sized crRNA and reactivate Cas12a for the second trans-cleavage reaction, which leads to an additional fluorescence signal amplification (FIG. 1b). Thus, the asymmetric CRISPR assay provides a simple, rapid, highly sensitive nucleic acid detection in one-pot by a single CRISPR-Cas12a.

Competitive CRISPR Reaction of Full-Sized crRNA and Split crRNA

To investigate the binding affinity of different crRNAs to CRISPR-Cas12a, a target-specific full-sized crRNA and a target-specific split crRNA that specifically binds to the same region of a DNA target were designed. In addition, two competitors (full-sized crRNA and split crRNA) that bind to a different target sequence than the target-specific full-sized crRNA and split crRNA were designed (FIG. 2a). The trans-cleavage activity of the target-specific split crRNA and full-sized crRNA at different concentrations of competitor full-sized crRNA (10, 20, 40, and 80 nM) was compared. It was observed that the trans-cleavage activity by the target-specific split crRNA significantly decreased as the concentration of the competitor full-sized crRNA increased. By contrast, the trans-cleavage reaction by the target-specific full-sized crRNA was not significantly affected as the concentration of the competitor full-sized crRNA increased (FIG. 2b). Next, the effect of the competitor split crRNA on the trans-cleavage activity of the target-specific split crRNA and full-sized crRNA for DNA target detection was determined. The competitor split crRNA did not significantly inhibit the trans-cleavage reactions of either the target-specific full-sized crRNA or the target-specific split crRNA (FIG. 2b). Based on these results, it was hypothesized that the full-sized crRNA has a stronger binding affinity to Cas12a than the split crRNA.

To test the hypothesis, the interaction between Cas12a and crRNAs was compared and visualized using an electrophoretic mobility shift assay. First, Cy5-labeled full-sized crRNA and FAM-labeled split crRNA (handle) were designed. Next, the two types of crRNAs were mixed at different concentration ratios with Cas12a to form a Cas12/crRNA complex. As shown in FIG. 2c, the full-sized crRNA was highly bound to Cas12a even when the concentration of the split crRNA increased (left). On the contrary, the split crRNA lost its ability to bind to Cas12a as the concentration of the full-sized crRNA increased (right). These findings further support the fluorescence testing results caused by the trans-cleavage activity of CRISPR-Cas12a (FIG. 2b). Thus, the full-sized crRNA can regulate the split crRNA binding to Cas12a and generate a competitive CRISPR reaction due to its stronger binding affinity.

Cascade Signal Amplification by Competitive crRNA

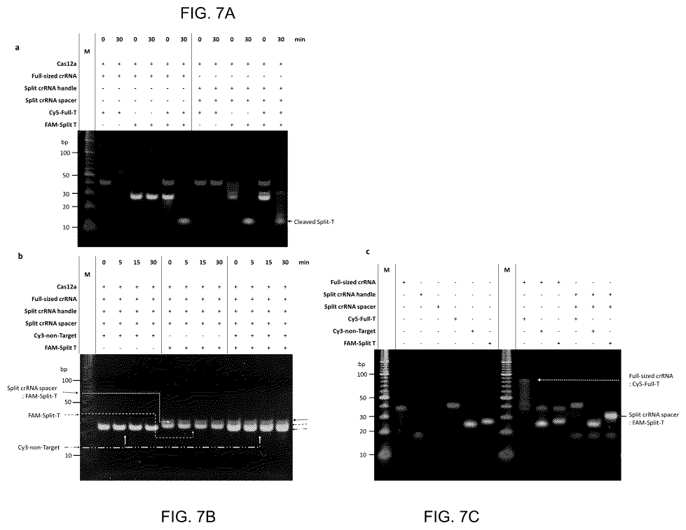

By taking advantage of the difference in affinity to Cas12a between the full-sized crRNA and split crRNA, the cascade signal amplification mechanism for highly sensitive nucleic acid detection was explored by conformational reactivation of CRISPR-Cas12a. In the asymmetric CRISPR assay, a larger amount of high-affinity full-sized crRNA was used than split crRNA. Thus, the affinity binding reaction with Cas12a by the full-sized crRNA proceeded dominantly and strongly while the reaction by the split crRNA was inhibited by the full-sized crRNA. It was confirmed that the Cy5-full-T (target of full-sized crRNA) cleavage could occur by full-sized crRNA regardless of split crRNAs (Lanes 2-5, FIG. 3a); however, in the case of FAM-split-T (target of split crRNA), the cleavage reaction by split crRNA was inhibited in the presence of full-sized crRNA (Lanes 6-9, FIG. 3a). Also, it was further confirmed that FAM-split-T could be cleaved by split crRNA in the absence of the full-sized crRNA (FIG. 7a). These results indicate that the full-sized crRNA reaction predominately occurs than the split crRNA reaction in the presence of both, which is consistent with the finding of the competitive CRISPR reaction between the two types of crRNAs (FIG. 2).

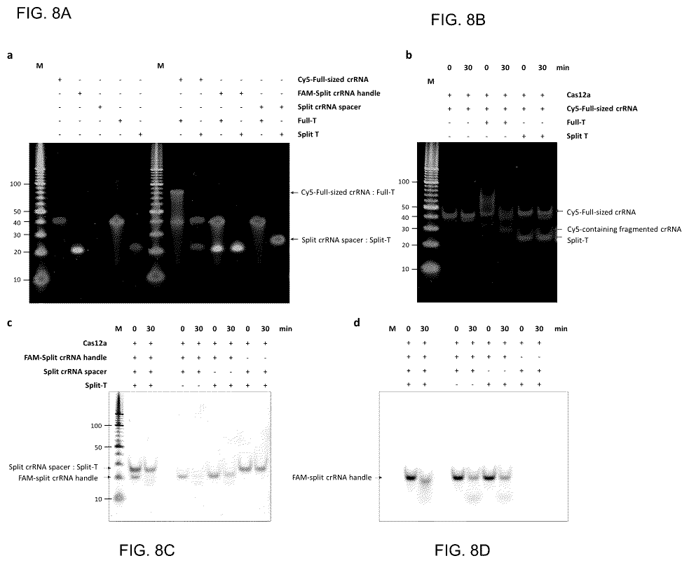

To better explain the mechanism of the competitive CRISPR reaction, Cy5-full-sized crRNA and FAM-split crRNA were used to investigate the CRISPR reaction. In the presence of full-T, it was observed that the hybrid structure of Cy5-full-sized crRNA and full-T gradually degraded, resulting in the generation of full-sized crRNA fragments containing Cy5 (Lanes 5 and 9, FIG. 3b). Also, it was found that the 5′ end cleavage of crRNA even in the absence of target sequences (FIG. 3b and FIG. 8). Meanwhile, in the absence of full-T, the split crRNA reaction with Cas12a was initially suppressed by the full-sized crRNA despite the presence of split-T (Lanes 6 and 7, FIG. 3b). However, in the presence of full-T, a decrease in the signal of the FAM-split crRNA was observed, which was thought to be due to the pre-crRNA processing by Cas12a (Lanes 5 and 9, FIG. 3b). In previous literature, a conformational resetting mechanism has been proposed to explain that new crRNA can replace the crRNA-DNA hybrid or the cleaved R-loop after the CRISPR-Cas12a cleavage reaction (Stella S, et al. Conformational Activation Promotes CRISPR-Cas12a Catalysis and Resetting of the Endonuclease Activity. Cell 175, 1856-1871 e1821 (2018)). Thus, without wishing to be bound by this theory, it is hypothesized that the split crRNA could replace the full-sized crRNA and bind to Cas12a after the activation of full-sized crRNA, enabling a cascade signal amplification reaction of CRISPR.

A competition assay was performed in which pre-activated Cas12a/Cy5-full-sized crRNA, Cas12a/Cy5-full-sized crRNA/non-target, and Cas12a/Cy5-full-sized crRNA/full-T complexed were incubated with the increasing concentration of FAM-split crRNA. As shown in FIG. 3c, in the absence of the full-T or in the presence of non-target, the Cy5-full-sized crRNA remained bound to Cas12a and could not be displaced by split crRNA even with increasing concentration of split crRNA. Interestingly, FAM-split crRNA could bind to Cas12a under the condition of pre-activated Cas12a/Cy5-full-sized crRNA/full-T, which shows that the split crRNA can reset and activate Cas12a by replacing the hybrid of the full-sized crRNA and the full-T in Cas12a. In other words, when full-sized crRNA and split crRNA were mixed, the split crRNA reaction with Cas12a was initially suppressed by full-sized crRNA despite the presence of split-T. When the target nucleic acid of full-sized crRNA, full-T, was added, Cas12a was activated and triggered the first trans-cleavage reaction with full-sized crRNA. Then, the split crRNA could replace the full-sized crRNA and bind with Cas12a, reactivating Cas12a through its split-T and inducing a second trans-cleavage reaction, resulting in cascade signal amplification.

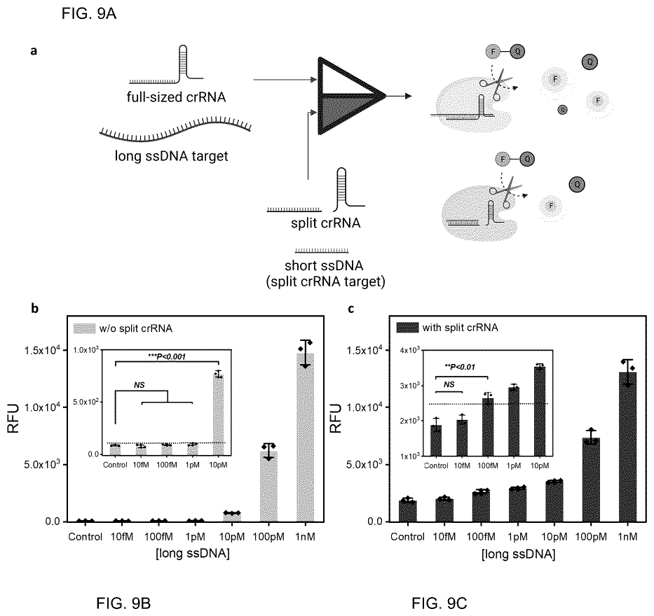

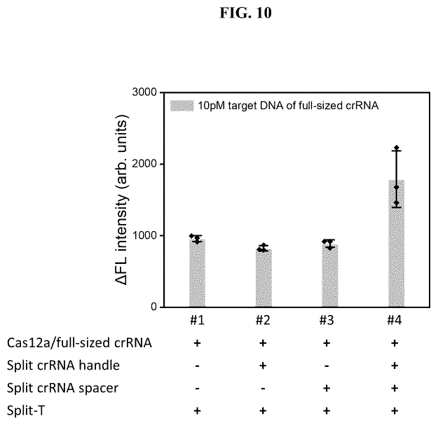

Next, the fluorescence signal of target DNA was measured at various concentrations by both the conventional CRISPR assay and the asymmetric CRISPR assay. As with the previous competitive crRNA reaction, it was observed that the trans-cleavage reaction of the split crRNA for split-T decreased in the presence of the full-sized crRNA. Interestingly, when comparing the DNA target detection efficiency in the presence or the absence of the split crRNA and split-T, the detection efficiency was improved when the split crRNA and split-T were added (FIG. 9). While, when only either split crRNA handle or spacer was added, the enhanced fluorescence signal was not observed (FIG. 10). In the case of DNA target detection, the asymmetric CRISPR assay detected the ssDNA target with a limit of detection (LOD) of 100 fM, which was 100 times more sensitive than that of the conventional CRISPR-Cas12a assay (FIG. 9). Thus, by leveraging the unique Cas12a kinetic difference caused by the competition reaction between two structurally different crRNAs, an asymmetric CRISPR assay for highly sensitive nucleic acid detection was developed.

### Example 2—RNA Detection of Cas12a Using Fragmented RNA/DNA Target

Cas12a/crRNA is known to catalyze trans-ssDNA cleavage after binding to a crRNA-complementary ssDNA as well as a dsDNA containing a PAM sequence. For direct detection of RNA by CRISPR-Cas12a in the asymmetric CRISPR assay, two fragmented nucleic acid targets that specifically bind crRNA and unleash indiscriminate trans-ssDNA cleavage activity of Cas12a (FIG. 4a). The trans-cleavage reaction was investigated after assembling Cas12a/crRNA with an ssDNA activator and ssRNA/ssDNA target in different combinational fashions (FIG. 4b-d). The trans-cleavage activity cannot occur when only the ssDNA activator or ssRNA/ssDNA target is present. Interestingly, the ssDNA 5′-activator/ssDNA 3′-target and ssRNA 5′-target/ssDNA 3′-activator were able to induce the collateral cleavage of Cas12a, whereas the ssDNA 5′-activator/ssRNA 3′-target could not. This result could be attributed to the weak binding affinity of the ssRNA 3′-target with crRNA compared with the ssDNA 3′-activator when it is targeted to the crRNA's seed region. Recently, several studies reported that a seed region that is 5-10 nucleotides (nt) distances from the PAM within the protospacers is important for target recognition of Cas12a. In addition, a similar finding was reported that the PAM-proximal ‘seed’ region of crRNA strictly tolerates DNA for initiating trans-cleavage, and the PAM-distal region or 3′-end of the crRNA can tolerate both RNA and DNA substrates. Thus, the binding position of ssRNA with crRNA highly affects the trans-cleavage activity of Cas12a (FIG. 4a-d).

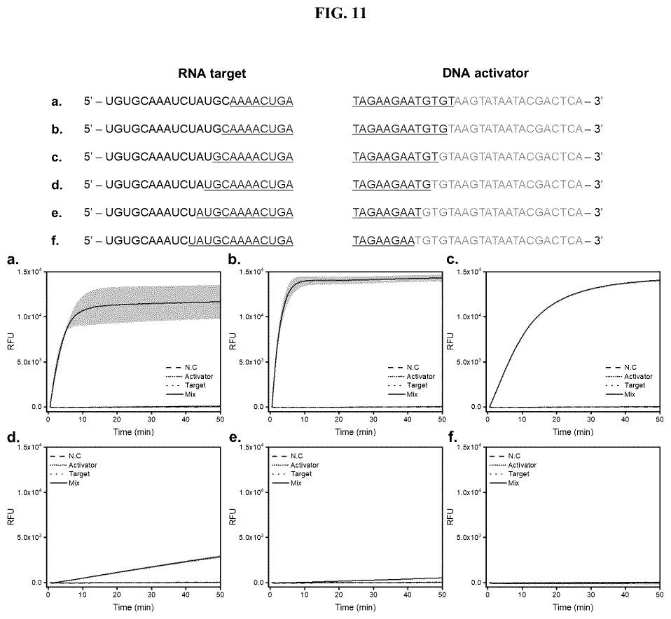

After validating the RNA detection ability of Cas12a by using fragmented RNA/DNA targets, attempts to apply it to detect miRNA were performed. As a proof of concept, miR-19a was as a target miRNA sequence because it is a potential biomarker for early diagnostics of many cancers. A universal ssDNA 3′-activator was designed to be complementary to the seed region of the crRNA. To compare and optimize miR-19a detection, a series of crRNAs were designed to bind ssDNA 3′-activator/miR-19a 5′-target with a single-nucleotide difference (FIG. 11). The crRNA that binds to miRNA 5′-target and ssDNA 3′-activator is hereafter referred to as RD crRNA. As shown in FIG. 11, the binding length between RD crRNA and ssDNA 3′-activator was highly related to the trans-cleavage activity of Cas12a. As the binding length between RD crRNA and ssDNA 3′-activator increased, the trans-cleavage reaction was strongly induced; however, it was reduced as the binding length decreased to 8-9 nt.

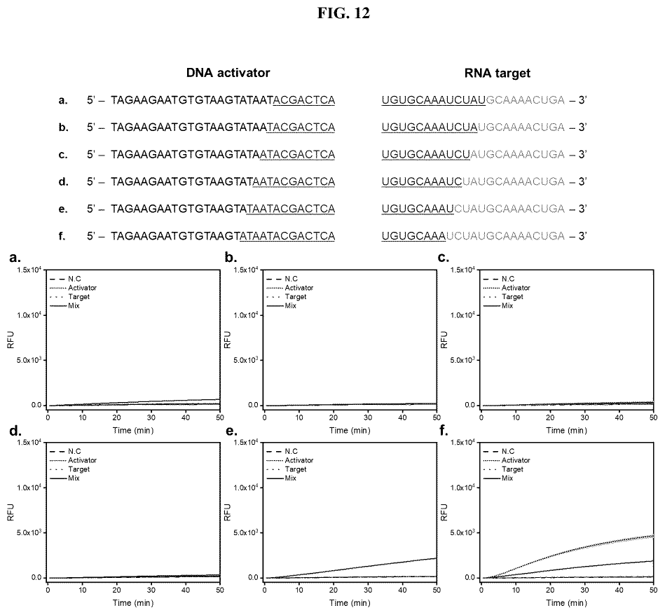

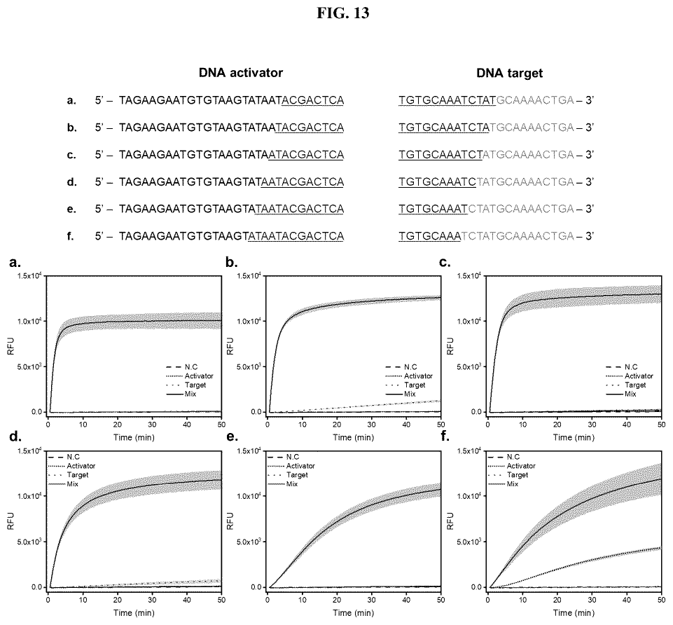

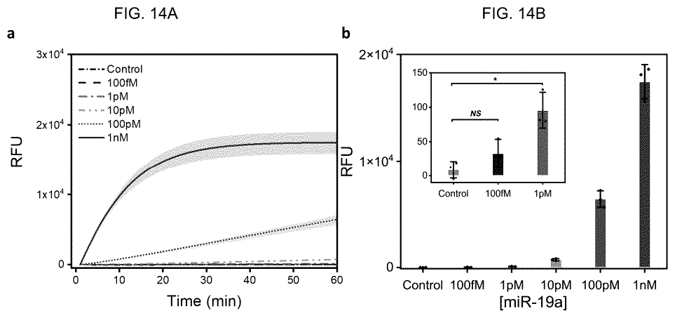

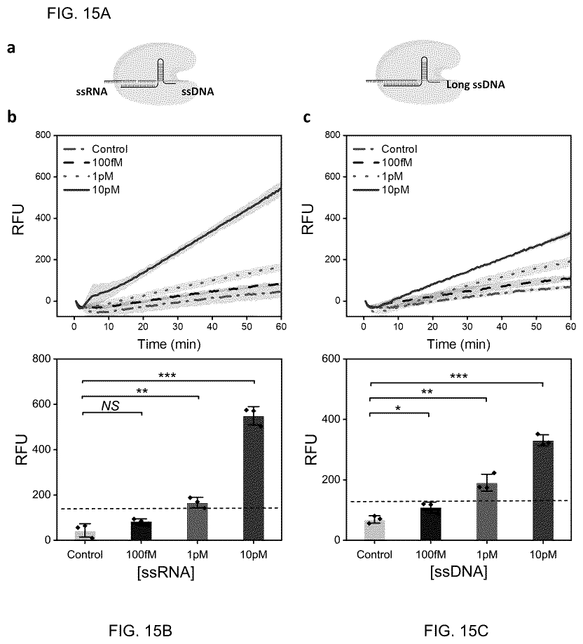

For comparison, a miR-19a 3′-target/ssDNA 5′-activator was designed by reversing the targeted locations of the RD crRNA and designed crRNA that binds to ssDNA 5′-activator and miR-19a 3′-target (DD crRNA). Interestingly, they could not initiate the collateral cleavage reaction of Cas12a regardless of the crRNA binding position (FIG. 12). By contrast, when both target nucleic acids were ssDNA, an efficient cleavage reaction was observed regardless of the binding length (FIG. 13). Based on this finding, the miRNA was placed at the 5′-target position close to the 3′ end of the crRNA. In addition, an RD crRNA was designed to bind to 9 nt of the miRNA and to 12 nt of the DNA activator. Furthermore, the detection sensitivity of Cas12a for miRNA detection was tested by using fragmented RNA/DNA targets. The real-time fluorescence signal was measured depending on various concentrations of target miRNA (100 fM˜1 nM) and confirmed that a 1 pM level of miRNA could be detected (FIG. 14). When compared to the detection efficiency of a full-sized ssDNA target containing miRNA and DNA activator sequences, there was no significant difference between the ssDNA target and the miRNA/DNA hybrid targets (FIG. 15). This finding also indicates that the RNA target could initiate the trans-cleavage activity of Cas12a by using a fragmented RNA/DNA target strategy and achieved a similar degree as the conventional full-sized ssDNA target.

### Example 3—Amplification-Free miRNA Quantitative Detection

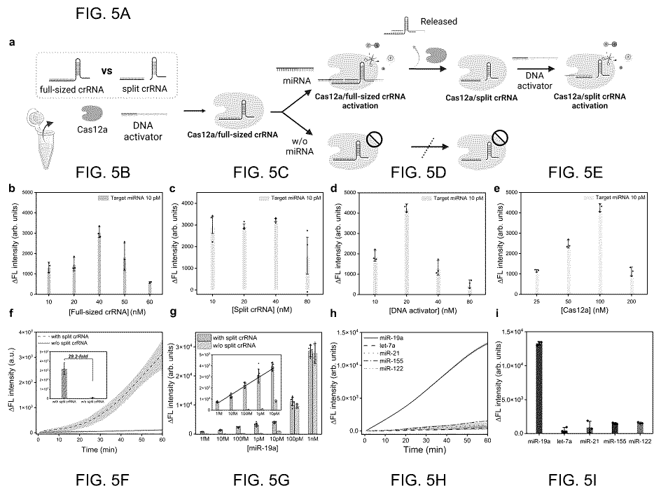

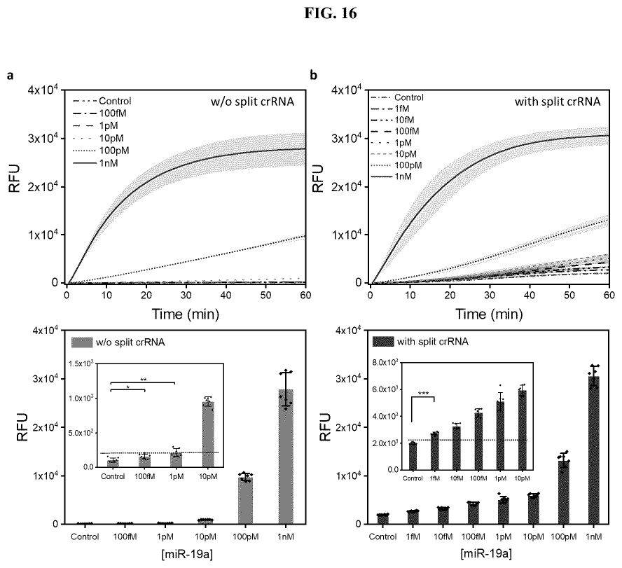

Next, whether further improvement of the miRNA detection sensitivity of Cas12a was investigated by applying the exemplary asymmetric CRISPR assay, enabling amplification-free miRNA detection. To eliminate the need for an additional split crRNA target, a ssDNA 3′-activator that simultaneously can serve as the split crRNA target was designed Here, a split crRNA was designed to recognize part of the DNA activator (portion of the DNA activator, FIG. 5a), along with a full-sized crRNA that simultaneously binds to the miRNA (underlined portion of the miRNA, FIG. 5a) and the DNA activator (underlined portion of the DNA activator, FIG. 5a). To determine the optimal conditions for the asymmetric CRISPR assay, the concentration of full-sized crRNA and split crRNA was tested (FIGS. 5b and 5c). The fluorescence signal was measured from each target sample and control sample, and then the ΔFL intensity was obtained by subtracting the fluorescence signal of the control sample from the fluorescence signal of the target sample. Since the split crRNA reaction is inhibited by the full-sized crRNA, the fluorescence signal was not changed significantly with the change in split crRNA concentrations ranging from 10 to 40 nM. In the case of full-sized crRNA, the most optimal results were observed at a concentration of 40 nM. As shown in FIG. 5b-e, the conditions for obtaining optimal detection performance were determined to be 100 nM for LbCas12a, 40 nM for full-sized crRNA, 10 nM for split crRNA, and 20 nM for DNA activator. When testing different targets, the reaction condition can be further optimized due to the potential existence of the secondary or tertiary structure of different target sequences. Under the optimized reaction conditions, real-time fluorescence signals for miRNA detection were monitored at various concentrations ranging from 1 fM to 1 nM. Interestingly, in the high-concentration target miRNA condition, there was no significant difference compared with the CRISPR detection without split crRNA; however, a notable difference in fluorescence signal emerged in the low-concentration target condition (FIG. 5f and FIG. 5g, and FIG. 16). For instance, the endpoint fluorescence signal difference of the 1 pM target miRNA in the asymmetric CRISPR assay was 29.2 times higher than that in the absence of split crRNA (FIG. 5f).

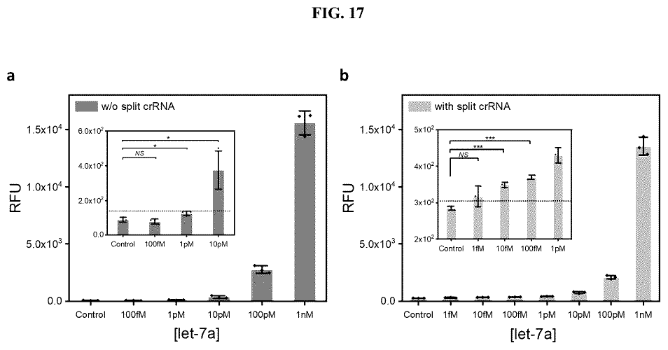

As shown in FIG. 5g, ΔFluorescence intensity (F_{target miRNA}−F_{control}), which is the difference in fluorescence signal between the control and target miRNA, was improved by adding split crRNA, and the detection sensitivity was significantly improved compared to the case without split crRNA. In particular, the fluorescence intensity linearly increased with the logarithm concentration of miR-19a ranging from 1 fM to 10 pM in the presence of split crRNA. With the asymmetric CRISPR assay, a LOD of 856 aM of miR-19a was achieved, which is 1,000 times more sensitive than the CRISPR detection in the absence of split crRNA (FIG. 16, Table 2). This detection sensitivity is comparable to combined CRISPR-based miRNA detection methods with pre-amplification step. To demonstrate the detection versatility of the assay, let-7a was also tested, another cancer miRNA biomarker, and obtained a similar result (FIG. 17). Next, to evaluate the selectivity of the developed assay, several types of miRNAs (let-7a, miR-21, miR155, and miR-122) were tested and compared. A strong fluorescence increase was observed with the target miR-19a; however, other types of miRNAs were unable to enhance the fluorescence signal, indicating the high specificity of this assay (FIG. 5h and FIG. 5i). Thus, the asymmetric CRISPR assay provides a simple, highly sensitive, and specific approach for amplification-free, quantitative detection of miRNA.

TABLE 2
Summary of the ratio of positive tests in asymmetric CRISPR assay
to detect the target miRNA. The probit analysis demonstrates that
the 95% detection limit of the asymmetric CRISPR assay is 856 aM.

| | |
|---|---|
| Target miRNA | No. of positive tests / |
| concentration | No. of total tests |

| | | |
|---|---|---|
| 1 | fM | 15/15 |
| 500 | aM | 10/15 |
| 200 | aM | 7/15 |
| 100 | aM | 0/15 |
| 50 | aM | 0/15 |

### Example 4—Clinical Validation of the Asymmetric CRISPR Assay

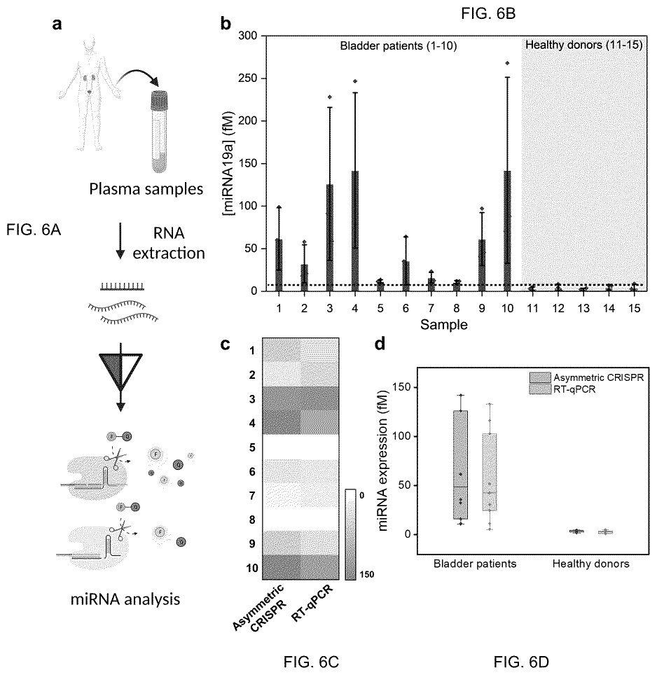

Finally, the potential utility of the asymmetric CRISPR assay for miRNA liquid biopsy in cancer diagnosis and prognosis was explored. miRNAs have been implicated in the development and progression of various types of cancer. miRNA liquid biopsies that detect changes in miRNA expression levels in bodily fluids (e.g., blood, urine, or saliva) can provide important diagnostic and prognostic information about diseases and are more convenient and patient-friendly than tissue biopsies. In particular, studies have shown that miR-19a is overexpressed in bladder cancer patients, contributing to the growth and metastasis of bladder cancer. Here, the expression level of miR-19a was analyzed in plasma samples collected from patients with bladder cancer using the asymmetric CRISPR assay (FIG. 6a).

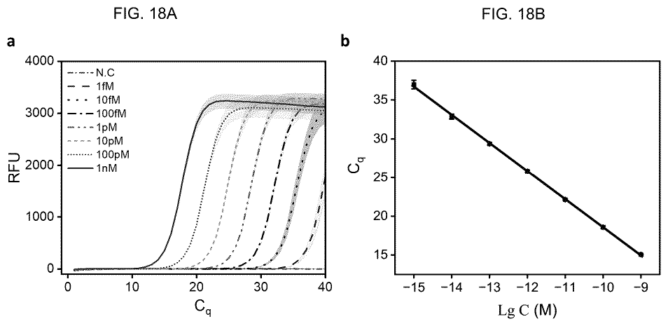

First, total RNA was extracted from plasma samples collected from ten bladder cancer patients and five healthy donors using a commercial RNA extraction kit. The extracted total RNA was mixed with a reaction solution including Cas12a/crRNA and split crRNA, then measured the fluorescence signal in real-time. Subsequently, the concentration of the target miR-19a in each plasma sample was estimated based on the standard curve that was obtained using the relationship between miRNA concentration and the ΔFluorescence intensity (F_{target miRNA}−F_{control}). As a result, it was confirmed that the overall expression level of miR-19a was higher in the bladder patient samples compared to the healthy donor samples (FIG. 6b and FIG. 6d). Concurrently, the same extracted RNA samples were analyzed using reverse-transcription quantitative PCR (RT-qPCR). A standard curve was constructed (FIG. 18) based on the relationship between miRNA concentration and the Cq values of the RT-qPCR. Then, the concentration of miR-19a in each plasma sample was calculated based on the standard curve of the RT-qPCR. The developed assay showed an excellent correlation with the results using RT-qPCR as shown in FIGS. 6c and 6d. Together, these clinical testing results demonstrate that the asymmetric CRISPR method enables a simple, rapid, and amplification-free detection of miRNA in clinical samples with high sensitivity, providing empirical support for its clinical application.

### Materials & Methods

All oligonucleotide sequences used in this study were obtained from Integrated DNA Technologies (IDT, USA) and are listed in Table 3. LbCas12 (Cpf1, Cat #M0653), 10×NEBuffer 2.1 (Cat #B6002S), and proteinase K (Cat #P8107S) were purchased from New England Biolabs (NEB, USA). RNase-free water (Cat #11-05-01-14) was purchased from IDT. The 10×TBE (tris borate EDTA) buffer (Cat #1610770), 10×TAE (tris acetate EDTA) buffer (Cat #161-0743), 40% acrylamide/bis solution (Cat #1610146), ammonium persulfate (Cat #1610700), and TEMED (Cat #1610800) were purchased from Bio-Rad (USA). SYBR gold nucleic acid gel stain was purchased from Invitrogen (USA). Human plasma samples from healthy donors were purchased from Innovative Research (USA). Human plasma samples from bladder cancer patients were collected and provided by the Carole and Ray Neag Comprehensive Cancer Center with a protocol approved by the ethics committee at the University of Connecticut Health Center (IRB #08-310-1) (Table 4). The miRNeasy Serum/Plasma Advanced Kit (Cat #217204), miRCURY LNA RT Kit (Cat #339340), miRCURY LNA SYBR Green PCR Kit (Cat #339345), and has-miR-19a-3p miRCURY LNA miRNA PCR assay (Cat #339306) were purchased from Qiagen (USA).

TABLE 3
Oligonucleotide sequences

| Name | Sequence (5′→3′) | bp |
|---|---|---|
| RD crRNA 8 | /AltR1/rUrA rArUrUrUrCrU rArCrU rArArG rUrGrU rArGrA rUrUrU rCrUrU | 42 |
| | rCrUrA rUrCrA rGrUrU rUrUrG rCrArU rA/AltR2/ (SEQ ID NO: 1) | |
| RD crRNA 9 | /AltR1/rUrA rArUrU rUrCrU rArCrU rArArG rUrGrU rArGrA rUrArU rUrCrU | 42 |
| | rUrCrU rArUrC rArGrU rUrUrU rGrCrA rU/AltR2/ (SEQ ID NO: 2) | |
| RD crRNA 10 | /AltR1/rUrA rArUrU rUrCrU rArCrU rArArG rUrGrU rArGrA rUrCrA rUrUrC | 42 |
| | rUrUrC rUrArU rCrArG rUrUrU rUrGrC rA/AltR2/ (SEQ ID NO: 3) | |
| RD crRNA 11 | /AltR1/rUrA rArUrU rUrCrU rArCrU rArArG rUrGrU rArGrA rUrArC rArUrU | 42 |
| | rCrUrU rCrUrA rUrCrA rGrUrU rUrUrG rC/AltR2/ (SEQ ID NO: 4) | |
| RD crRNA 12 | /AltR1/rUrA rArUrU rUrCrU rArCrU rArArG rUrGrU rArGrA rUrCrA rCrArU | 42 |
| | rUrCrU rUrCrU rArUrC rArGrU rUrUrU rG/AltR2/ (SEQ ID NO: 5) | |
| RD crRNA 13 | /AltR1/rUrA rArUrU rUrCrU rArCrU rArArG rUrGrU rArGrA rUrArC rArCrA | 42 |
| | rUrUrC rUrUrC rUrArU rCrArG rUrUrU rU/AltR2/ (SEQ ID NO: 6) | |
| DD crRNA 8 | /AltR1/rUrA rArUrU rUrCrU rArCrU rArArG rUrGrU rArGrA rUrUrU rUrGrC | 42 |
| | rArCrA rUrGrA rGrUrC rGrUrA rUrUrA rU/AltR2/ (SEQ ID NO: 7) | |
| DD crRNA 9 | /AltR1/rUrA rArUrU rUrCrU rArCrU rArArG rUrGrU rArGrA rUrArU rUrUrG | 42 |
| | rCrArC rArUrG rArGrU rCrGrU rArUrU rA/AltR2/ (SEQ ID NO: 8) | |
| DD crRNA 10 | /AltR1/rUrA rArUrU rUrCrU rArCrU rArArG rUrGrU rArGrA rUrGrA rUrUrU | 42 |
| | rGrCrA rCrArU rGrArG rUrCrG rUrArU rU/AltR2/ (SEQ ID NO: 9) | |
| DD crRNA 11 | /AltR1/rUrA rArUrU rUrCrU rArCrU rArArG rUrGrU rArGrA rUrArG rArUrU | 42 |
| | rUrGrC rArCrA rUrGrA rGrUrC rGrUrA rU/AltR2/ (SEQ ID NO: 10) | |
| DD crRNA 12 | /AltR1/rUrA rArUrU rUrCrU rArCrU rArArG rUrGrU rArGrA rUrUrA rGrArU | 42 |
| | rUrUrG rCrArC rArUrG rArGrU rCrGrU rA/AltR2/ (SEQ ID NO: 11) | |
| DD crRNA 13 | AltR1/rUrA rArUrU rUrCrU rArCrU rArArG rUrGrU rArGrA rUrArU rArGrA | 42 |
| | rUrUrU rGrCrA rCrArU rGrArG rUrCrG rU/AltR2/ (SEQ ID NO: 12) | |
| miR-19a | UGU GCA AAU CUA UGC AAA ACU GA (SEQ ID NO: 13) | 23 |
| miR-19a DNA | TGT GCA AAT CTA TGC AAA ACT GA (SEQ ID NO: 14) | 23 |
| Let7a crRNA | /AltR1/rUrA rArUrU rUrCrU rArCrU rArArG rUrGrU rArGrA rUrCrA | 42 |
| | rCrArU rUrCrU rUrCrU rArArA rCrUrA rUrArC rA/AltR2/ (SEQ ID NO: 15) | |
| Let-7a | UGA GGU AGU AGG UUG UAU AGU U (SEQ ID NO: 16) | 22 |
| Let-7b | rUrGrA rGrGrU rArGrU rArGrG rUrUrG rUrGrU rGrGrU rU (SEQ ID NO: 17) | 22 |
| Let-7c | rUrGrA rGrGrU rArGrU rArGrG rUrUrG rUrArU rGrGrU rU (SEQ ID NO: 18) | 22 |
| miR-21 | UAG CUU AUC AGA CUG AUG UUG A (SEQ ID NO: 19) | 22 |
| miR-155 | UUA AUG CUA AUC GUG AUA GGG GUU (SEQ ID NO: 20) | 24 |
| miR-122 | UGG AGU GUG ACA AUG GUG UUU G (SEQ ID NO: 21) | 22 |
| Universal DNA | TAG AAG AAT GTG TAA GTA TAA TAC GAC TCA (SEQ ID NO: 22) | 30 |
| probe | | |
| FAM reporter | /56-FAM/TT ATT /3IABKFQ/ | 5 |
| RD DNA target | TGT GCA AAT CTA TGC AAA ACT GAT AGA AGA ATG TGT AAG TAT | 53 |
| (Long ssDNA) | AAT ACG ACT CA (SEQ ID NO: 23) | |
| crRNA handle | rUrArA rUrUrU rCrUrA rCrUrA rArGrU rGrUrA rGrArU (SEQ ID NO: 24) | 21 |
| crRNA spacer | rUrGrA rGrUrC rGrUrA rUrUrA rUrArC rUrUrA rCrArC (SEQ ID NO: 25) | 21 |
| Target-specific | /AltR1/rUrA rArUrU rUrCrU rArCrU rArArG rUrGrU rArGrA rUrUrG rArGrU | 42 |
| full-sized crRNA | rCrGrU rArUrU rArUrA rCrUrU rArCrA rC/AltR2/ (SEQ ID NO: 26) | |
| Competitor full- | /AltR1/rUrA rArUrU rUrCrU rArCrU rArArG rUrGrU rArGrA rUrCrA | 42 |
| sized crRNA | rCrArU rUrCrU rUrCrU rArUrC rArGrU rUrUrU rG/AltR2/ (SEQ ID NO: 27) | |
| Competitor split | rArArC rUrArU rArCrA rArCrC rUrArC rUrArC rCrUrC rA (SEQ ID NO: 28) | 22 |
| crRNA spacer | | |
| Split crRNA | TGA GGT AGT AGG TTG TAT AGT T (SEQ ID NO: 29) | 22 |
| target ssDNA | | |
| Cy5-full sized | /5Cy5/rUrA rArUrU rUrCrU rArCrU rArArG rUrGrU rArGrA rUrCrA rCrArU | 42 |
| crRNA | rUrCrU rUrCrU rArUrC rArGrU rUrUrU rG (SEQ ID NO: 30) | |
| FAM-split | /56-FAM/rUrA rArUrU rUrCrU rArCrU rArArG rUrGrU rArGrA rU (SEQ ID | 21 |
| crRNA handle | NO: 31) | |
| Cy5-full-T | /5Cy5/TG TGC AAA TCT ATG CAA AAC TGA TAG AAG AAT GTG TAA | 53 |
| | GTA TAA TAC GAC TCA (SEQ ID NO: 32) | |
| FAM-split-T | /56-FAM/AA ATG AGG TAG TAG GTT GTA TAG TT (SEQ ID NO: 33) | 25 |
| Cy3-non-target | /5Cy3/GA TGT ACA AAT ATC CAG TGG AAC TTC ACT TTT G (SEQ ID | 33 |
| | NO: 34) | |

TABLE 4
Bladder cancer patient sample information. Age range is 45-78.

| | Gender | Diagnosis |
|---|---|---|
| 1 | M | High-grade T2-2 papillary |
| 2 | F | Focal papillary and focal in situ urothelial carcinoma, grade 3/3 |
| 3 | M | High-grade urothelial papillary transitional cell carcinoma, grade 3/3 |
| 4 | M | Exophytic, papillary transitional cell carcinoma, grade 1 with scattered |
| | | foci grade 2/3 |
| 5 | M | High-grade urothelial (transitional cell) papillary carcinoma |
| 6 | M | High-grade invasive urothelial carcinoma |
| 7 | M | Focus of high-grade urothelial carcinoma |
| 8 | M | Urothelial (transitional cell) carcinoma, foci of high grade within |
| | | background of low grade |
| 9 | M | Urothelial (transitional cell) carcinoma, pTa, grade 2/3 |
| 10 | M | High-grade urothelial carcinoma, pT1 |

Competitive CRISPR Reaction Assay Between the Full-Sized crRNA and Split crRNA

2 μL LbCas12a (1 μM), 0.2 μL target-specific full-sized crRNA (1 μM) (0.2 μL each of 1 M target-specific split crRNA handle and spacer), 0.2 μL of different concentrations of competitor full-sized crRNA (0.2 μL each of competitor split crRNA handle and spacer), 1 μL fluorescence-quencher probe (10 μM) and 2 μL 10×NEBuffer r2.1 buffer was mixed with 2 μL ssDNA target (10 nM) and added RNase-free water to a final volume of 20 μL. For the control sample, 2 μL RNase-free water was added instead of the ssDNA target. Each fluorescence signal of the target and control sample was measured in real-time at 37° G using a GFX96 touch real-time PGR system (Bio-Rad, GA, USA).

### Conventional Cas12a/crRNA Assay

The cleavage reaction was conducted in a final volume of 20 μL, including 2 μL 10×NEBuffer r2.1 buffer, 2 μL LbCas12a (1 pM), 0.2 μL crRNA (1 pM), 1 μL fluorescence-quencher probe (10 pM), 12.8 μL RNase-free water and 2 μL various concentrations of target ssDNA. The fluorescence signal was measured at 30-s intervals at 37° C. using a CFX96 touch real-time PCR system (Bio-Rad, CA, USA).

### Fragmented Nucleic Acid Target Detection Using Cas12a/crRNA

The cleavage reaction was conducted in a final volume of 20 μL, including 2 μL 10×NEBuffer r2.1 buffer, 2 μL LbCas12a (1 μM), 0.2 μL crRNA (1 μM), 2 μL DNA activator (100 nM), 1 μL fluorescence-quencher probe (10 μM), 10.8 μL RNase-free water and 2 μL various concentrations of ssRNA/ssDNA target. The fluorescence signal was measured at 30-s intervals at 37° C. using a CFX96 touch real-time PCR system (Bio-Rad, CA, USA).

Asymmetric CRISPR Assay for miRNA Detection

The cleavage reaction was conducted in a final volume of 20 μL, including 2 μL 10×NEBuffer r2.1 buffer, 2 μL LbCas12a (1 μM), 0.8 μL full-sized crRNA (1 μM), 0.2 μL each split crRNA (1 μM handle and spacer), 0.4 μL DNA activator (1 μM), 2 μL fluorescence-quencher probe (10 PM), 10.4 μL RNase-free water, and 2 μL various concentrations of target miRNA. The fluorescence signal was measured at 30-s intervals at 37° C. using a CFX96 touch real-time PCR system.

Asymmetric CRISPR Assay for Long ssDNA Detection

The cleavage reaction was conducted in a final volume of 20 μL, including 2 μL 10×NEBuffer r2.1 buffer, 2 μL LbCas12a (1 μM), 0.8 μL full-sized crRNA (1 μM), 0.2 μL each split crRNA (1 μM handle and spacer), 0.1 μL ssDNA target of split crRNA (1 μM), 1 μL fluorescence-quencher probe (10 μM), 11.7 μL RNase-free water, and 2 μL various concentrations of target ssDNA. The fluorescence signal was measured at 30-s intervals at 37° C. using a CFX96 touch real-time PCR system.

### Gel Electrophoresis

For the test using fluorophore-conjugated crRNA, a total of 20 μL of the reaction solution was prepared by mixing 200 nM LbCas12a, 100 nM crRNA (full-sized and split crRNA), and different combinations of 100 nM full-sized crRNA target (full-T) and 50 nM split crRNA target (split-T) in 1×NEBuffer 2.1 buffer and nuclease-free water. After incubation of the reaction solution at 37° C. for 0 and 30 min, proteinase K was added for 15 min at 45° C. Then, each reaction solution was resolved on a 10% polyacrylamide gel using 1×TBE as the running buffer at a constant voltage of 100 V for 100 min. Gels were scanned using a ChemiDoc Imaging System (Bio-Rad).

For the test using fluorophore-conjugated target nucleic acid, a total of 20 μL of the reaction solution was prepared by mixing 100 nM LbCas12a, 40 nM full-sized crRNA, 10 nM split crRNA, and different combinations of 100 nM Cy5-conjugated full-T/Cy3-conjugated non-target and 50 nM FAM-conjugated split-T in 1×NEBuffer 2.1 buffer and nuclease-free water. After incubation of the reaction solution at 37° C. for 0, 5, 15, and 30 min, each reaction solution was resolved on a 10% polyacrylamide gel using 1×TBE as the running buffer at a constant voltage of 100 V for 100 min. Gels were scanned using a ChemiDoc Imaging System (Bio-Rad).

### Electrophoretic Mobility Shift Assay

Fluorescence-labeled crRNAs (5′-Cy5-conjugated full-sized crRNA and 5′-FAM-conjugated split crRNA handle) were mixed in different concentrations. Two groups were prepared: (1) Cy5-full-sized crRNA was fixed at 200 nM and the concentration of the FAM-split crRNA handle varied from 0, 50, 100, 200, and 400 nM. (2) FAM-split crRNA handle was fixed at 200 nM and the concentration of the Cy5-full-sized crRNA varied from 0, 50, 100, 200, and 400 nM. Next, the prepared crRNA was mixed with 250 nM LbCas12a in 1×NEBuffer 2.1 to prepare a total reaction solution of 20 μL. After incubation for 20 min at 25° C., the reaction solutions were loaded onto a 5% polyacrylamide gel using 1×TAE as the running buffer at a constant voltage of 100 V for 100 min. Gels were scanned using the ChemiDoc Imaging System (Bio-Rad).

For the competitive assay, three groups were prepared: (1) 250 nM LbCas12a and 200 nM Cy5-full-sized crRNA, (2) 250 nM LbCas12a, 200 nM Cy5-full-sized crRNA, and 100 nM non-target sequence, and (3) 250 nM LbCas12a, 200 nM Cy5-full-sized crRNA, and 100 nM target sequence. Then, the prepared samples were incubated at 37° C. for 20 min. After incubation, different concentrations of FAM-split crRNA were added (0, 50, 100, 200, and 400 nM) and incubated at 25° C. for 20 min. Next, the reaction solutions were loaded onto a 5% polyacrylamide gel using 1×TAE as the running buffer at a constant voltage of 100 V for 100 min. Gels were scanned using the ChemiDoc Imaging System (Bio-Rad).

miRNA Detection Using Human Plasma Samples

Total RNA was extracted from the human plasma samples using the miRNeasy Serum/Plasma Advanced Kit (Qiagen), according to the manufacturer's instructions. The extracted total RNA was diluted 5-fold before testing with the Cas12a assay. Next, the RNA solution was mixed with the asymmetric CRISPR assay reaction components including 1×NEBuffer 2.1 buffer, 100 nM LbCas12a, 40 nM crRNA, 10 nM each split crRNA (handle and spacer), 20 nM DNA activator, and 1 μM fluorescence-quencher probe. The fluorescence signal was measured at 30-s intervals at 37° C. using a CFX96 touch real-time PCR system.

### RNA Extraction and RT-qPCR

Total RNA was extracted from the human plasma samples by using the miRNeasy Serum/Plasma Advanced Kit (Qiagen), according to the manufacturer's instructions. cDNA was synthesized from the extracted total RNA using the miRCURY LNA RT Kit (Qiagen). Briefly, a reverse-transcription reaction was performed at 42° C. for 60 min and then inactivated at 95° C. for 5 min. Synthesized cDNA was stored at −4° C. before use. The synthesized cDNA was amplified using the miRCURY LNA SYBR Green PCR Kit and has-miR-19a-3p miRCURY LNA miRNA PCR assay (Qiagen) following the manufacturer's protocol. Total reaction solution contained 5 μL of 2×SYBR green master mix, 1 μL of PCR primer mix (has-miR-19a-3p miRCURY LNA miRNA PCR assay (Qiagen)), 1 μL of RNase-free water, and 3 μL of cDNA template. PCR was performed with 40 cycles of 95° C. for 10 s and 56° C. for 60 s.

Embodiments disclosed here are not limiting of the subject matter and is merely exemplary. Various other components may be included and called upon for providing for aspects of the teachings herein. For example, additional materials, combinations of materials and/or omission of materials may be used to provide for added embodiments that are within the scope of the teachings herein.

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

The term “binding region” refers to a region of a first polynucleotide that is complementary to and binds to a portion of a second polynucleotide. In the context of the present disclosure, the binding regions are stretches of nucleotides in a crRNA or scrRNA that are complementary to and bind to portions of target nucleic acids. The target nucleic acid can be an endogenous nucleic acid that is present within a sample or cell or an exogenous nucleic acid that is introduced into a sample or cell, such as a ssDNA probe. The disclosure contemplates the use of crRNAs comprising first and second binding regions that are complementary to and bind to different portions of a target nucleic acid and a ssDNA probe.

In some instances, the first binding region comprises a portion of the ′3 end of the at least one crRNA that is complementary to a portion of the 3′ end of a 5′ target strand of the target nucleic acid and a second binding region comprising a portion of the 5′ end that is complementary to a portion of the 5′ region of a 3′ target strand of the at least one ssDNA probe. The first binding region between the at least one crRNA and the target nucleic acid can comprise a length of from about 6 to about 15 nucleotides and the second binding region between the at least one crRNA and the at least one ssDNA probe comprises a length of from about 15 to about 6 nucleotides. In some embodiments, the first binding region has a length of about 6, about 7, about 8, about 9, about 10, about 11, about 12, about 13, about 14, or about 15 nt and the second binding region comprises a length of about 15, about 14, about 13, about 12, about 11, about 10, about 9, about 8, about 7, or about 6 nt. Preferably, the total length of combined first and second binding regions of the at least one crRNA is about 21 nucleotides and as the first binding region increases in length by one nucleotide the second binding region decreases in length by one nucleotide.

In other instances, the first binding region comprises a portion of the ′3 end of the at least one crRNA that is complementary to a portion of the 5′ end of a 3′ target strand of the target nucleic acid and a second binding region comprising a portion of the 5′ end that is complementary to a portion of the 3′ region of a 5′ target strand of the at least one ssDNA probe. The first binding region between the at least one crRNA and the target nucleic acid can comprise a length of from about 15 to about 6 nucleotides and the second binding region between the at least one crRNA and the at least one ssDNA probe can comprise a length of from about 6 to about 15 nucleotides. In some embodiments, the first binding region has a length of about 15, about 14, about 13, about 12, about 11, about 10, about 9, about 8, about 7, or about 6 nt and the second binding region comprises a length of about 6, about 7, about 8, about 9, about 10, about 11, about 12, about 13, about 14, or about 15 nt. Preferably, the total length of combined first and second binding regions of the at least one crRNA is about 21 nucleotides and as the first binding region decreases in length by one nucleotide the second binding region increases in length by one nucleotide.

In some instances, the at least one scrRNA comprises a binding region that is complementary to a portion of the 3′ end of a 3′ target strand of the at least one ssDNA probe. The binding region of the at least one scrRNA can comprise a length of from about 17 to about 24 nucleotides. In some embodiments, the binding region of the at least one scrRNA comprises a length of about 17, about 18, about 19, about 20, about 21, about 22, about 23, or about 24 nt. Preferably, the length of the binding region between the at least one scrRNA and the at least one ssDNA probe increases by one nucleotide as the length of the second binding region between the at least one crRNA and the at least one ssDNA probe decreases in length by one nucleotide.

In other instances, the at least one scrRNA comprises a binding region that is complementary to a portion of the 5′ end of a 5′ target strand of the at least one ssDNA probe. The binding region of the at least one scrRNA can comprise a length of from about 24 to 17 nucleotides. In some embodiments, the binding region of the at least one scrRNA comprises a length of about 24, about 23, about 22, about 21, about 20, about 19, about 18, or about 17 nt. Preferably, the length of the binding region between the at least one scrRNA and the at least one ssDNA probe decreases by one nucleotide as the length of the second binding region between the at least one crRNA and the at least one ssDNA probe increases in length by one nucleotide.

As used herein, “CRISPR-RNA (crRNA)” refers to a short RNA sequence that guides a CRISPR-Cas protein to a complementary target nucleic acid sequence (such as DNA or RNA) within a sample. The crRNA forms part of the CRISPR-Cas complex and is responsible for the sequence-specific recognition of the target nucleic acid, enabling precise binding and, initiation of a trans-cleavage reaction by the CRISPR-Cas protein\crRNA complex. In the asymmetric amplification systems and methods of the present disclosure, crRNA is configured to recognize and specifically bind to nucleic acid sequences associated with the presence of pathogens, genetic mutations, or other markers of interest, triggering a detectable signal that can then be amplified by a detectable signal that is generated when a scrRNA of the present disclosure initiates a second trans-cleavage reaction of a CRISPR-Cas protein\scrRNA complex. The total length of the crRNA, including binding regions, can vary. Preferably, the total length of the crRNA is from about 41 to about 44 nucleotides (nts). In some embodiments, the total length of the crRNA is about 41, about 42, about 43, or about 44 nt.

As used herein “detectable label” refers to any chemical, biochemical, or molecular moiety that can be attached to a CRISPR-associated nucleic acid (e.g., at least one crRNA, at least one scrRNA, or at least one ssDNA probe, etc.) to enable the detection, identification, or quantification of a target nucleic acid. The label generates a measurable signal upon binding, hybridization, cleavage, or interaction with the target sequence in a sample. Detectable labels may include, but are not limited to, radionuclides, fluorophores such as fluorescein, rhodamine, Texas Red, Cy2, Cy3, Cy5, and the AlexaFluor® (Invitrogen, Carlsbad, Calif.) range of fluorophores, chromophores, quantum dots, antibodies, gadolinium, gold, nanomaterials, horseradish peroxidase, alkaline phosphatase, derivatives thereof, and mixtures thereof, or any other molecule that produces a detectable signal using appropriate instrumentation, such as fluorescence microscopy, flow cytometry, or spectroscopy. The choice of label will depend on the detection method and assay sensitivity required for the CRISPR nucleic acid detection assay.

As used herein, “optional” or “optionally” means that the subsequently described event or circumstance may or may not occur, and that the description includes instances where said event or circumstance occurs and instances where it does not.

The term “sample” is used herein in the broadest sense and refers to any suitable sample, including liquids, solids, and gases. In some embodiments, the sample is a biological sample (e.g., a sample obtained from a subject). The biological sample may comprise a fluid sample or a tissue sample. In some embodiments, the biological sample is a blood sample or a blood product such as serum or plasma. In some embodiments, the sample comprises urine. In embodiments, the sample is a respiratory specimen, including a nasal sample (e.g., a nasal swab), a nasopharyngeal sample (e.g., a nasopharyngeal swab), an oropharyngeal sample (e.g., an oropharyngeal swab), a mid-turbinate sample (e.g., a midturbinate swab), sputum, endotracheal aspirate or bronchoalveolar lavage. In some embodiments, the sample is a cerebrospinal fluid sample. In some embodiments, the sample is a saliva sample. In some embodiments, the sample is a tissue sample. In some embodiments, the sample is obtained from a subject suspected of having a viral infection. In some embodiments, the sample is obtained from a subject suspected of having an upper respiratory infection. In some embodiments, the sample is obtained from a subject suspected of having a SARS-CoV-2 infection. In some embodiments, the subject is a human. The sample can be used directly as obtained from a patient or can be pre-treated, such as by heating, filtration, distillation, extraction, concentration, centrifugation, inactivation of interfering components, addition of reagents, and the like.

As used herein, “split CRISPR-RNA (scrRNA)” refers to an intact CRISPR-RNA (crRNA) that has been split into two separate fragments, including a 5′ handle region and a 3′ spacer region. The scrRNAs can be programmed to target any nucleic acid and amplify a signal generated by a first trans-cleavage reaction resulting from a CRISPR-Cas protein/crRNA complex. The total length of the scrRNA including the combined 5′ handle region and 3′ spacer can vary, as can the length of the 5′ handle region and the ′3 spacer region. Preferably, the total length of the scrRNA is from about 40 to about 44 nt. In some embodiments, the length of the scrRNA is about 40 nt. In other embodiments, the length of the scrRNA is about 41 nt. In yet other embodiments, the length of the scrRNA is about 42 nt. In still other embodiments, the length of the scrRNA is about 43 nt. In further embodiments, the length of the scrRNA is about 41 nt. Preferably, the length of the 5′ handle region is from about 19 to about 20 nt. In some embodiments, the length of the 5′ handle region is about 19 nt. In other embodiments, the length of the 5′ handle region is about 20 nt. Preferably, the length of the 3′ spacer region is from about 21 nt to about 24 nt. In some embodiments, the length of the 3′ spacer region is about 21 nt. In other embodiments, the length of the 3′ spacer region is about 22 nt. In yet other embodiments, the length of the 3′ spacer region is about 23 nt. In still other embodiments, the length of the 3′ spacer region is about 24 nt.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments.

The terms “target sequence,” “target nucleic acid,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a Cas/CRISPR complex, provided sufficient conditions for binding exist. In some embodiments, the target sequence is a viral nucleic acid sequence. In an embodiment, the target sequence includes SARS-CoV-2, influenza virus, drug-resistant influenza viruses, human immunodeficiency virus (HIV), and/or a combination thereof. In an embodiment, the target sequence includes several types of biomarkers such as miRNA, mRNA, circulating cell-free DNA (cfDNA), RNA (cfRNA), and/or a combination thereof. In an embodiment, the target sequence is a SARS-CoV-2 sequence. In an embodiment, the target sequence is a human immunodeficiency virus (HIV).

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

A significant change is any detectable change that is statistically significant in a standard parametric test of statistical significance such as Student's t-test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims.

For reasons of completeness, various aspects of the disclosure are set out in the following numbered clauses:

Clause 1. A CRISPR signal amplification system for detecting a target nucleic acid, comprising: (a) a CRISPR-Cas protein; (b) at least one crisprRNA (crRNA) configured to specifically bind to a first target nucleic acid, wherein the at least one crRNA comprises: (i) a first detectable label; and (ii) a first binding affinity to the CRISPR-Cas protein; (c) at least one split crRNA (scrRNA) configured to specifically bind to a second target nucleic acid other than the first target nucleic acid, wherein the at least one scrRNA comprises: (i) a crRNA that is split into a 5′ handle region and a 3′ spacer region; (ii) a second detectable label; and (iii) a second binding affinity to the CRISPR-Cas protein that is weaker than the first binding affinity of the crRNA to the CRISPR-Cas protein; wherein when the at least one crRNA, the at least one scrRNA are present in a reaction mixture with the CRISPR-Cas protein and the first and second target nucleic acids, the at least one crRNA outcompetes the at least one scrRNA to form a Cas protein/crRNA complex that is activated in the presence of the first target nucleic acid to initiate a first trans-cleavage reaction resulting in replacement of the at least one crRNA and release of the first detectable label to produce a first signal, and wherein upon completion of the first trans-cleavage reaction and consumption of the at least one crRNA, the at least one scrRNA forms a Cas protein/scrRNA complex that is activated in the presence of the second target nucleic acid to initiate a second trans-cleavage reaction resulting in replacement of the at least one scrRNA and release of the second detectable label to produce a second signal, thereby amplifying the first signal.

Clause 2. A method for asymmetric signal amplification for detecting a target nucleic acid in a sample, the method comprising: (a) reacting an effective amount of a CRISPR-Cas protein, at least one crRNA, and at least one split crRNA in a sample comprising or suspected of comprising at least a first target nucleic acid for a period of time sufficient for the Cas12a protein to initiate a first trans-cleavage reaction involving a Cas12a/crRNA complex and a second trans-cleavage reaction involving a Cas protein/scrRNA complex, wherein: (i) the at least one crisprRNA (crRNA) is configured to specifically bind to the first target nucleic acid, and comprises: (1) a first detectable label; and (2) a first binding affinity to the CRISPR-cas12a protein; (ii) the at least one split crRNA (scrRNA) is configured to specifically bind to a second target nucleic acid other than the first target nucleic acid and comprises: (1) a crRNA that is split into a 5′ handle region and a 3′ spacer region; (2) a second detectable label; and (3) a second binding affinity to the CRISPR-cas12a protein that is weaker than the first binding affinity of the crRNA to the CRISPR-cas12a protein; wherein when the at least one crRNA, the at least one scrRNA are present in the sample with the CRISPR-cas12a protein and the first and second target nucleic acids, the at least one crRNA outcompetes the at least one scrRNA to form a Cas protein/crRNA complex that is activated in the presence of the first target nucleic acid to initiate the first trans-cleavage reaction resulting in cleavage of the at least one crRNA and release of the first detectable label to produce a first signal, and wherein upon completion of the first trans-cleavage reaction and consumption of the at least one crRNA, the at least one scrRNA forms a Cas protein/scrRNA complex that is activated in the presence of the second target nucleic acid to initiate the second trans-cleavage reaction resulting in cleavage of the at least one scrRNA and release of the second detectable label to produce a second signal, thereby amplifying the first signal; and (b) measuring the first and second signal produced in the sample in step (a); and (c) detecting the target nucleic acid in the sample based on the first and second signal measured in step (b).

Clause 3. An asymmetric amplification system for detecting target nucleic acids comprising: (a) a CRISPR-Cas protein; (b) at least one single stranded DNA (ssDNA) probe comprising a 5′ region with a first detectable label and a 3′ region with a second detectable label; (c) at least one crisprRNA (crRNA) configured to simultaneously and specifically bind to the 5′ region of the ssDNA probe and a target nucleic acid, wherein the at least one crRNA comprises a first binding affinity to the CRISPR-Cas protein; (d) at least one split crRNA (scrRNA) configured to specifically bind to the 3′ region of the ssDNA probe, wherein the scrRNA comprises: (i) a crRNA that is split into a 5′ handle region and a 3′ spacer region; and (ii) a second binding affinity to the CRISPR-Cas protein that is weaker than the first binding affinity of the crRNA to the CRISPR-Cas protein; wherein when the at least one crRNA and the at least one scrRNA are present in a reaction with the CRISPR-Cas protein, the at least one ssDNA probe, and the target nucleic acid, the at least one crRNA simultaneously binds to the 5′ region of the ssDNA probe and the target nucleic acid and outcompetes the at least one scrRNA to form a Cas protein/crRNA complex that is activated in the presence of the target nucleic acid to initiate a first trans-cleavage reaction resulting in cleavage of the at least one crRNA and release of the first detectable label to produce a first signal, and wherein upon completion of the first trans-cleavage reaction and consumption of the crRNA, the at least one scrRNA forms a Cas protein/scrRNA complex that is activated in the presence of the at least one ssDNA probe to initiate a second trans-cleavage reaction resulting in cleavage of the at least one scrRNA and release of the second detectable label to produce a second signal, thereby amplifying the first signal.

Clause 4. A cascade signal amplification method for detecting a target nucleic acid in a sample, the method comprising: (a) reacting an effective amount of a CRISPR-Cas protein, at least one crRNA, at least one split crRNA, and at least one ssDNA probe in a sample comprising or suspected of comprising at least a first target nucleic acid for a period of time sufficient for the Cas12a protein to initiate a first trans-cleavage reaction involving a Cas12a/crRNA complex and a second trans-cleavage reaction involving a Cas protein/scrRNA complex, wherein: (i) the at least one single stranded DNA (ssDNA) probe comprises a 5′ region with a first detectable label and a 3′ region with a second detectable label; (ii) the at least one crisprRNA (crRNA) is configured to simultaneously and specifically bind to the 5′ region of the ssDNA probe and the first target nucleic acid and comprises a first binding affinity to the CRISPR-Cas protein; (iii) the at least one split crRNA (scrRNA) is configured to specifically bind to the 3′ region of the ssDNA probe and comprises: (1) a crRNA that is split into a 5′ handle region and a 3′ spacer region; and (2) a second binding affinity to the CRISPR-cas12a protein that is weaker than the first binding affinity of the crRNA to the CRISPR-cas12a protein; wherein when the at least one crRNA and the at least one scrRNA are present in the sample with the CRISPR-Cas protein, the at least one ssDNA probe, and the first target nucleic acid, the at least one crRNA simultaneously binds to the 5′ region of the ssDNA probe and the target nucleic acid and outcompetes the at least one scrRNA to form a Cas protein/crRNA complex that is activated in the presence of the target nucleic acid to initiate the first trans-cleavage reaction resulting in cleavage of the at least one crRNA and release of the first detectable label to produce a first signal, and wherein upon completion of the first trans-cleavage reaction and consumption of the crRNA, the at least one scrRNA forms a Cas protein/scrRNA complex that is activated in the presence of the at least one ssDNA probe to initiate the second trans-cleavage reaction resulting in cleavage of the at least one scrRNA and release of the second detectable label to produce a second signal, thereby amplifying the first signal; (b) measuring the first and second signal produced in the sample in step (a); and (c) detecting the target nucleic acid in the sample based on the first and second signal measured in step (b).

Clause 5. The system of clause 3 or method of clause 4, wherein the at least one crRNA comprises a first binding region comprising a portion of the ′3 end of the at least one crRNA that is complementary to a portion of the 3′ end of a 5′ target strand of the target nucleic acid and a second binding region comprising a portion of the 5′ end that is complementary to a portion of the 5′ region of a 3′ target strand of the at least one ssDNA probe.

Clause 6. The system or method of clause 5, wherein the first binding region between the at least one crRNA and the target nucleic acid comprises a length of from about 6 to about 15 nucleotides and the second binding region between the at least one crRNA and the at least one ssDNA probe comprises a length of from about 15 to about 6 nucleotides.

Clause 7. The system or method of clause 6, wherein the total length of combined first and second binding regions of the at least one crRNA is about 21 nucleotides and as the first binding region increases in length by one nucleotide the second binding region decreases in length by one nucleotide.

Clause 8. The system of clause 3 or method of clause 4, wherein the at least one crRNA comprises a first binding region comprising a portion of the ′3 end of the at least one crRNA that is complementary to a portion of the 5′ end of a 3′ target strand of the target nucleic acid and a second binding region comprising a portion of the 5′ end that is complementary to a portion of the 3′ region of a 5′ target strand of the at least one ssDNA probe.

Clause 9. The system or method of clause 8, wherein the first binding region between the at least one crRNA and the target nucleic acid comprises a length of from about 15 to about 6 nucleotides and the second binding region between the at least one crRNA and the at least one ssDNA probe comprises a length of from about 6 to about 15 nucleotides.

Clause 10. The system or method of clause 9, wherein the total length of combined first and second binding regions of the at least one crRNA is about 21 nucleotides and as the first binding region decreases in length by one nucleotide the second binding region increases in length by one nucleotide.

Clause 11. The system or method of any one of clauses 3-10, wherein the: (i) at least one scrRNA comprises a binding region that is complementary to a portion of the 3′ end of a 3′ target strand of the at least one ssDNA probe; or (ii) at least one scrRNA comprises a binding region that is complementary to a portion of the 5′ end of a 5′ target strand of the at least one ssDNA probe.

Clause 12. The system or method of clause 11, wherein (i) wherein the binding region comprises a length of from about 17 to about 24 nucleotides; (ii) wherein the binding region comprises a length of from about 24 to 17 nucleotides.

Clause 13. The system or method of clause 12, wherein: (i) the length of the binding region between the at least one scrRNA and the at least one ssDNA probe increases by one nucleotide as the length of the second binding region between the at least one crRNA and the at least one ssDNA probe decreases in length by one nucleotide; or (ii) the length of the binding region between the at least one scrRNA and the at least one ssDNA probe decreases by one nucleotide as the length of the second binding region between the at least one crRNA and the at least one ssDNA probe increases in length by one nucleotide.

Clause 14. The system of any one of clauses 1, 3 and 5-13 or method of any one of clauses 2 and 4-13, wherein an amount of the at least one crRNA is greater than an amount of the at least one scrRNA.

Clause 15. The system of clause 14, wherein the amount of the at least one crRNA is greater than the amount of the at least one scrRNA by a factor of at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 75-fold, or at least 100-fold.

Clause 16. The system of any one of clauses 1, 3, 5-15, or method of any one of clauses 2, and 4-15, wherein the first and second detectable labels comprise a fluorophore.

Clause 17. The system or method of clause 16, wherein the fluorophore comprises a fluorescent dye.

Clause 18. The system of any one of clauses 1, 3 and 5-17 or method of any one of clauses 2 and 4-17 wherein the limit of detection of the method is at least 50 fM, at least 55 fM, at least 60 fM, at least 65 fM, at least 70 fM, at least 75 fM, at least 80 fM, at least 85 fM, at least 90 fM, at least 95 fM, or at least 100 fM.

Clause 19. The system or method of clause 18, wherein the limit of detection is from 10 to 100 times more sensitive than a conventional CRISPR-Cas12a assay lacking at least one split scrRNA.

Clause 20. The system of any one of clauses 1, 3, and 5-18 or method of any one of clauses 2 and 4-19, wherein the target nucleic acid comprises RNA.

Clause 21. The system or method of clause 20, wherein the RNA comprises miRNA.

Clause 22. The system or method of clause 21, wherein the miRNA comprises miR-19a, miR-21, miR-23, miR-122, miR-126, miR-146a, miR-155, miR, miR-191, or mir200a.

Clause 22. The system of any one of clauses 1, 3 and 5-21 or method of any one of clauses 2 and 4-21, wherein the crRNA has a length of from about 41 to about 44 nucleotides

Clause 23. The system of any one of clauses 1, 3 and 5-22 or method of any one of clauses 2 and 4-22, wherein the scrRNA has a length of from about 40 to about 44, wherein the 5′ handle region is from about 19 to about 20 nucleotides and the 3-spacer region is from about 21 to about 24 nucleotides.

Those of ordinary skill in the art will appreciate that various changes and modifications to this description may be made without departing from the spirit or scope of the present invention, as defined in the following claims.

What is claimed is:

1. An asymmetric CRISPR amplification system, comprising:
(a) a CRISPR-Cas protein;
(b) at least one crisprRNA (crRNA) configured to specifically bind to a first target nucleic acid; and
(c) at least one split crRNA (scrRNA) configured to specifically bind to a second target nucleic acid other than the first target nucleic acid.

2. The system of claim 1, wherein the at least one crRNA comprises
(i) a first detectable label; and
(ii) a first binding affinity to the CRISPR-cas protein.

3. The system of claim 2, wherein the at least one scrRNA comprises:
(i) a crRNA that is split into a 5′ handle region and a 3′ spacer region;
(ii) a second detectable label; and
(iii) a second binding affinity to the CRISPR-Cas protein that is weaker than the first binding affinity of the crRNA to the CRISPR-Cas protein.

4. The system of claim 3, wherein when the at least one crRNA, the at least one scrRNA are present in a reaction mixture with the CRISPR-Cas protein and the first and second target nucleic acids, the at least one crRNA outcompetes the at least one scrRNA to form a Cas protein/crRNA complex that is activated in the presence of the first target nucleic acid to initiate a first trans-cleavage reaction resulting in replacement of the at least one crRNA and release of the first detectable label to produce a first signal.

5. The system of claim 4, wherein upon completion of the first trans-cleavage reaction and consumption of the at least one crRNA, the at least one scrRNA forms a Cas protein/scrRNA complex that is activated in the presence of the second target nucleic acid to initiate a second trans-cleavage reaction resulting in replacement of the at least one scrRNA and release of the second detectable label to produce a second signal, thereby amplifying the first signal.

6. A method for asymmetric CRISPR amplification to detect a target nucleic acid in a sample, the method comprising:
(a) reacting an effective amount of a CRISPR-Cas protein, at least one crRNA, and at least one split crRNA in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex; and
(b) measuring the first and second signal produced in the sample in step (a); and
(c) detecting the target nucleic acid in the sample based on the first and second signal measured in step (b).

7. The method of claim 6, wherein the at least one crisprRNA (crRNA) is configured to specifically bind to the first target nucleic acid, and comprises:
(i) a first detectable label; and
(ii) a first binding affinity to the CRISPR-Cas protein.

8. The method of claim 7, wherein the at least one split crRNA (scrRNA) is configured to specifically bind to a second target nucleic acid other than the first target nucleic acid and comprises:
(i) a crRNA that is split into a 5′ handle region and a 3′ spacer region;
(ii) a second detectable label; and
(iii) a second binding affinity to the CRISPR-Cas protein that is weaker than the first binding affinity of the crRNA to the CRISPR-Cas protein.

9. The method of claim 8, wherein when the at least one crRNA, the at least one scrRNA are present in the sample with the CRISPR-Cas protein and the first and second target nucleic acids, the at least one crRNA outcompetes the at least one scrRNA to form a Cas protein/crRNA complex that is activated in the presence of the first target nucleic acid to initiate the first trans-cleavage reaction resulting in replacement of the at least one crRNA and release of the first detectable label to produce a first signal.

10. The method of claim 9, wherein upon completion of the first trans-cleavage reaction and consumption of the at least one crRNA, the at least one scrRNA forms a Cas protein/scrRNA complex that is activated in the presence of the second target nucleic acid to initiate the second trans-cleavage reaction resulting in replacement of the at least one scrRNA and release of the second detectable label to produce a second signal, thereby amplifying the first signal.

11. An asymmetric CRISPR amplification system, comprising:
(a) a CRISPR-Cas protein;
(b) at least one single stranded DNA (ssDNA) probe comprising a 5′ region with a first detectable label and a 3′ region with a second detectable label;
(c) at least one crisprRNA (crRNA) configured to simultaneously and specifically bind to the 5′ region of the ssDNA probe and a target nucleic acid; and
(d) at least one split crRNA (scrRNA) configured to specifically bind to the 3′ region of the ssDNA probe.

12. The system of claim 11, wherein the at least one crRNA comprises a first binding affinity to the CRISPR-Cas protein.

13. The system of claim 12, wherein the scrRNA comprises:
(i) a crRNA that is split into a 5′ handle region and a 3′ spacer region; and
(ii) a second binding affinity to the CRISPR-Cas protein that is weaker than the first binding affinity of the crRNA to the CRISPR-Cas protein.

14. The system of claim 13, wherein when the at least one crRNA and the at least one scrRNA are present in a reaction with the CRISPR-Cas protein, the at least one ssDNA probe, and the target nucleic acid, the at least one crRNA simultaneously binds to the 5′ region of the ssDNA probe and the target nucleic acid and outcompetes the at least one scrRNA to form a Cas protein/crRNA complex that is activated in the presence of the target nucleic acid to initiate a first trans-cleavage reaction resulting in replacement of the at least one crRNA and release of the first detectable label to produce a first signal.

15. The system of claim 14, wherein upon completion of the first trans-cleavage reaction and consumption of the crRNA, the at least one scrRNA forms a Cas protein/scrRNA complex that is activated in the presence of the at least one ssDNA probe to initiate a second trans-cleavage reaction resulting in replacement of the at least one scrRNA and release of the second detectable label to produce a second signal, thereby amplifying the first signal.

16. The system of claim 11, wherein the at least one crRNA comprises a first binding region comprising a portion of the ′3 end of the at least one crRNA that is complementary to a portion of the 3′ end of a 5′ target strand of the target nucleic acid and a second binding region comprising a portion of the 5′ end that is complementary to a portion of the 5′ region of a 3′ target strand of the at least one ssDNA probe.

17. A method for asymmetric CRISPR amplification to detect a target nucleic acid in a sample, the method comprising:
(a) reacting an effective amount of a CRISPR-Cas protein, at least one crRNA, at least one split crRNA, and at least one ssDNA probe in a sample comprising or suspected of comprising at least a first target nucleic acid for a period of time sufficient for the Cas protein to initiate a first trans-cleavage reaction involving a Cas protein/crRNA complex and a second trans-cleavage reaction involving a Cas protein/scrRNA complex;
(b) measuring the first and second signal produced in the sample in step (a); and
(c) detecting the target nucleic acid in the sample based on the first and second signal measured in step (b).

18. The method of claim 17, wherein:
(i) the at least one single stranded DNA (ssDNA) probe comprises a 5′ region with a first detectable label and a 3′ region with a second detectable label;
(ii) the at least one crisprRNA (crRNA) is configured to simultaneously and specifically bind to the 5′ region of the ssDNA probe and the first target nucleic acid and comprises a first binding affinity to the CRISPR-Cas protein; and
(iii) the at least one split crRNA (scrRNA) is configured to specifically bind to the 3′ region of the ssDNA probe and comprises:
(1) a crRNA that is split into a 5′ handle region and a 3′ spacer region; and
(2) a second binding affinity to the CRISPR-Cas protein that is weaker than the first binding affinity of the crRNA to the CRISPR-Cas protein.

19. The method of claim 18, wherein when the at least one crRNA and the at least one scrRNA are present in the sample with the CRISPR-Cas protein, the at least one ssDNA probe, and the first target nucleic acid, the at least one crRNA simultaneously binds to the 5′ region of the ssDNA probe and the target nucleic acid and outcompetes the at least one scrRNA to form a Cas protein/crRNA complex that is activated in the presence of the target nucleic acid to initiate the first trans-cleavage reaction resulting in replacement of the at least one crRNA and release of the first detectable label to produce a first signal.

20. The method of claim 19, wherein upon completion of the first trans-cleavage reaction and consumption of the crRNA, the at least one scrRNA forms a Cas protein/scrRNA complex that is activated in the presence of the at least one ssDNA probe to initiate the second trans-cleavage reaction resulting in replacement of the at least one scrRNA and release of the second detectable label to produce a second signal, thereby amplifying the first signal.


SEQUENCE LISTING (34 sequences)

SEQ ID NO: 1 - RNA, 84 nt, Synthetic construct
  source 1..84: mol_type = other RNA
rtrarartrtrtrcrtrarcrtrarargrtrgrtrargrartrtrtrcrtrtrcrtrartrcrargrtrtrtrtrgrcrartra

SEQ ID NO: 2 - RNA, 84 nt, Synthetic construct
  source 1..84: mol_type = other RNA
rtrarartrtrtrcrtrarcrtrarargrtrgrtrargrartrartrtrcrtrtrcrtrartrcrargrtrtrtrtrgrcrart

SEQ ID NO: 3 - RNA, 84 nt, Synthetic construct
  source 1..84: mol_type = other RNA
rtrarartrtrtrcrtrarcrtrarargrtrgrtrargrartrcrartrtrcrtrtrcrtrartrcrargrtrtrtrtrgrcra

SEQ ID NO: 4 - RNA, 84 nt, Synthetic construct
  source 1..84: mol_type = other RNA
rtrarartrtrtrcrtrarcrtrarargrtrgrtrargrartrarcrartrtrcrtrtrcrtrartrcrargrtrtrtrtrgrc

SEQ ID NO: 5 - RNA, 84 nt, Synthetic construct
  source 1..84: mol_type = other RNA
rtrarartrtrtrcrtrarcrtrarargrtrgrtrargrartrcrarcrartrtrcrtrtrcrtrartrcrargrtrtrtrtrg

SEQ ID NO: 6 - RNA, 84 nt, Synthetic construct
  source 1..84: mol_type = other RNA
rtrarartrtrtrcrtrarcrtrarargrtrgrtrargrartrarcrarcrartrtrcrtrtrcrtrartrcrargrtrtrtrt

SEQ ID NO: 7 - RNA, 84 nt, Synthetic construct
  source 1..84: mol_type = other RNA
rtrarartrtrtrcrtrarcrtrarargrtrgrtrargrartrtrtrtrgrcrarcrartrgrargrtrcrgrtrartrtrart

SEQ ID NO: 8 - RNA, 84 nt, Synthetic construct
  source 1..84: mol_type = other RNA
rtrarartrtrtrcrtrarcrtrarargrtrgrtrargrartrartrtrtrgrcrarcrartrgrargrtrcrgrtrartrtra

SEQ ID NO: 9 - RNA, 84 nt, Synthetic construct
  source 1..84: mol_type = other RNA
rtrarartrtrtrcrtrarcrtrarargrtrgrtrargrartrgrartrtrtrgrcrarcrartrgrargrtrcrgrtrartrt

SEQ ID NO: 10 - RNA, 84 nt, Synthetic construct
  source 1..84: mol_type = other RNA
rtrarartrtrtrcrtrarcrtrarargrtrgrtrargrartrargrartrtrtrgrcrarcrartrgrargrtrcrgrtrart

SEQ ID NO: 11 - RNA, 84 nt, Synthetic construct
  source 1..84: mol_type = other RNA
rtrarartrtrtrcrtrarcrtrarargrtrgrtrargrartrtrargrartrtrtrgrcrarcrartrgrargrtrcrgrtra

SEQ ID NO: 12 - RNA, 84 nt, Synthetic construct
  source 1..84: mol_type = other RNA
rtrarartrtrtrcrtrarcrtrarargrtrgrtrargrartrartrargrartrtrtrgrcrarcrartrgrargrtrcrgrt

SEQ ID NO: 13 - RNA, 23 nt, Synthetic construct
  source 1..23: mol_type = other RNA
tgtgcaaatctatgcaaaactga

SEQ ID NO: 14 - DNA, 23 bp, Synthetic construct
  source 1..23: mol_type = other DNA
tgtgcaaatctatgcaaaactga

SEQ ID NO: 15 - RNA, 84 nt, Synthetic construct
  source 1..84: mol_type = other RNA
rtrarartrtrtrcrtrarcrtrarargrtrgrtrargrartrcrarcrartrtrcrtrtrcrtrarararcrtrartrarcra

SEQ ID NO: 16 - RNA, 22 nt, Synthetic construct
  source 1..22: mol_type = other RNA
tgaggtagtaggttgtatagtt

SEQ ID NO: 17 - RNA, 44 nt, Synthetic construct
  source 1..44: mol_type = other RNA
rtrgrargrgrtrargrtrargrgrtrtrgrtrgrtrgrgrtrt

SEQ ID NO: 18 - RNA, 44 nt, Synthetic construct
  source 1..44: mol_type = other RNA
rtrgrargrgrtrargrtrargrgrtrtrgrtrartrgrgrtrt

SEQ ID NO: 19 - RNA, 22 nt, Synthetic construct
  source 1..22: mol_type = other RNA
tagcttatcagactgatgttga

SEQ ID NO: 20 - RNA, 24 nt, Synthetic construct
  source 1..24: mol_type = other RNA
ttaatgctaatcgtgataggggtt

SEQ ID NO: 21 - RNA, 22 nt, Synthetic construct
  source 1..22: mol_type = other RNA
tggagtgtgacaatggtgtttg

SEQ ID NO: 22 - DNA, 30 bp, Synthetic construct
  source 1..30: mol_type = other DNA
tagaagaatgtgtaagtataatacgactca

SEQ ID NO: 23 - DNA, 53 bp, Synthetic construct
  source 1..53: mol_type = other DNA
tgtgcaaatctatgcaaaactgatagaagaatgtgtaagtataatacgactca

SEQ ID NO: 24 - RNA, 42 nt, Synthetic construct
  source 1..42: mol_type = other RNA
rtrarartrtrtrcrtrarcrtrarargrtrgrtrargrart

SEQ ID NO: 25 - RNA, 42 nt, Synthetic construct
  source 1..42: mol_type = other RNA
rtrgrargrtrcrgrtrartrtrartrarcrtrtrarcrarc

SEQ ID NO: 26 - RNA, 84 nt, Synthetic construct
  source 1..84: mol_type = other RNA
rtrarartrtrtrcrtrarcrtrarargrtrgrtrargrartrtrgrargrtrcrgrtrartrtrartrarcrtrtrarcrarc

SEQ ID NO: 27 - RNA, 84 nt, Synthetic construct; identical to SEQ ID NO: 5
  source 1..84: mol_type = other RNA

SEQ ID NO: 28 - RNA, 44 nt, Synthetic construct
  source 1..44: mol_type = other RNA
rararcrtrartrarcrararcrcrtrarcrtrarcrcrtrcra

SEQ ID NO: 29 - DNA, 22 bp, Synthetic construct
  source 1..22: mol_type = other DNA
tgaggtagtaggttgtatagtt

SEQ ID NO: 30 - RNA, 84 nt, Synthetic construct; identical to SEQ ID NO: 5
  source 1..84: mol_type = other RNA

SEQ ID NO: 31 - RNA, 42 nt, Synthetic construct; identical to SEQ ID NO: 24
  source 1..42: mol_type = other RNA

SEQ ID NO: 32 - DNA, 53 bp, Synthetic construct; identical to SEQ ID NO: 23
  source 1..53: mol_type = other DNA

SEQ ID NO: 33 - DNA, 25 bp, Synthetic construct
  source 1..25: mol_type = other DNA
aaatgaggtagtaggttgtatagtt

SEQ ID NO: 34 - DNA, 33 bp, Synthetic construct
  source 1..33: mol_type = other DNA
gatgtacaaatatccagtggaacttcacttttg